(19) United States
(12) Patent Application Publication
(10) Pub. No.: US 2018/0137938 A1
(43) Pub. Date: May 17, 2018

(54) PATIENT MONITORING SYSTEMS AND METHODS WITH AUTOMATED DISPLAY TOGGLING

(71) Applicant: MEDTRONIC MINIMED, INC., Northridge, CA (US)

(72) Inventors: Santhisagar Vaddiraju, Plymouth, MN (US); Marina Kasamanian, Van Nuys, CA (US); Bejan M. Darbandi, Chanhassen, MN (US); Yi Guan, Santa Monica, CA (US); Hiten Chawla, Los Angeles, CA (US)

(21) Appl. No.: 15/847,784

(22) Filed: Dec. 19, 2017

Related U.S. Application Data
(63) Continuation-in-part of application No. 15/803,635, filed on Nov. 3, 2017.
(60) Provisional application No. 62/421,073, filed on Nov. 11, 2016.

(51) Int. Cl. G16H 40/63; G16H 10/60; G16H 80/00; A61B 5/00; A61B 5/145

(52) U.S. Cl. CPC G16H 40/63; G16H 10/60; G16H 80/00; H04L 67/141; A61B 5/14532; A61B 5/7445; A61B 5/0004

(57) ABSTRACT

Systems, devices and methods are provided for monitoring a physiological condition of a patient. An exemplary method involves a computing device obtaining measurement data for the physiological condition of the patient, providing a graphical user interface display pertaining to monitoring the physiological condition of the patient and identifying a toggling condition for the graphical user interface display. In response to the toggling condition, a graphical representation of at least some of the measurement data is automatically provided at the computing device. After providing the graphical representation of at least some of the measurement data, the computing device identifies a second toggling condition while the graphical representation of at least some of the measurement data is presented at the computing device and automatically removes the graphical representation of the at least some of the measurement data from presentation at the computing device in response to the second toggling condition.

### CROSS-REFERENCE TO RELATED APPLICATION(S)

This application is a continuation-in-part of U.S. patent application Ser. No. 15/803,635, filed Nov. 3, 2017, which claims the benefit of U.S. Provisional Patent Application Ser. No. 62/421,073, filed Nov. 11, 2016, the entire contents of which are incorporated by reference herein.

### TECHNICAL FIELD

Embodiments of the subject matter described herein relate generally to medical devices, and more particularly, embodiments of the subject matter relate to monitoring conditions that affect a patient's physiological condition or sensitivity to a fluid, medication, or other potential therapies or lifestyle modifications.

### BACKGROUND

Managing a diabetic's blood glucose level is complicated by variations in a user's daily activities (e.g., exercise, carbohydrate consumption, and the like) in addition to variations in the user's individual insulin response and potentially other factors. Physicians have recognized that continuous monitoring provides a greater understanding of a patient's glycemic profile, and accordingly, continuous glucose monitoring (CGM) is employed to gain insight into the patient's condition and make appropriate therapy and lifestyle recommendations to achieve improved glucose control. However, glucose measurements alone often do not provide sufficient context regarding a patient's daily activities and how those may be influencing the patient's glucose level. While patients can independently maintain a log or journal of their activities, integrating manual event logs and establishing appropriate temporal relationships with continuous glucose monitoring data can be time consuming and impose a burden on physicians and other healthcare providers, which given the limited time available to physicians, may discourage adoption and incorporation of continuous monitoring. Accordingly, there is a need provide continuous monitoring and integrated event log data to facilitate improved outcomes and minimize the burdens on patients, physicians, or other healthcare providers.

### BRIEF SUMMARY

Systems, devices and methods are provided for monitoring a physiological condition of a patient. One exemplary method of monitoring a physiological condition of a patient involves a computing device obtaining measurement data for the physiological condition of the patient, providing a graphical user interface display pertaining to monitoring the physiological condition of the patient and identifying a toggling condition for the graphical user interface display. In response to the toggling condition while the graphical user interface display hiding the measurement data is presented at the computing device, a graphical representation of at least some of the measurement data is automatically provided at the computing device. After providing the graphical representation of the measurement data, the computing device identifies a second toggling condition while the graphical representation of at least some of the measurement data is presented at the computing device and automatically removes the graphical representation of the measurement data from presentation at the computing device in response to identifying the second toggling condition.

In another embodiment, a monitoring system is provided. The monitoring system includes a monitoring device coupled to a sensing element to obtain measurement data pertaining to a physiological condition of a patient, a remote device coupled to a communications network, and a monitoring application at a client device coupled to the communications network. The monitoring application monitors a first network for a data ready indication from the monitoring device, establishes a communications session with the monitoring device on the first network in response to the data ready indication, receives the measurement data from the monitoring device over the first network, uploads the measurement data to the remote device over the communications network, and automatically toggles display of at least a portion of the measurement data at the monitoring device in response to detecting a toggling condition.

In another embodiment, a method of monitoring a physiological condition of a patient involves pairing a client computing device and a monitoring device over a personal area network, providing, at the client computing device, a home screen graphical user interface display pertaining to monitoring the physiological condition of the patient, and identifying, at the client computing device, a first display toggling condition while the home screen graphical user interface display is presented at the client computing device. The monitoring device is coupled to a sensing element obtaining measurement data indicative of the patient's physiological condition, which is hidden from the home screen graphical user interface display. In response to the first display toggling condition, the method continues by automatically establishing, by the client computing device, a communications session with the monitoring device on the personal area network, receiving, by the client computing device, the measurement data from the monitoring device via the communications session, and automatically providing, at the client computing device, a diagnostic display including a graphical representation of the measurement data in lieu of the home screen graphical user interface display. Thereafter, the method involves identifying, at the client computing device, a second display toggling condition while the diagnostic display is presented at the client computing device, and automatically presenting the home screen graphical user interface display in lieu of the diagnostic display at the client computing device in response to the second display toggling condition.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

### BRIEF DESCRIPTION OF THE DRAWINGS

A more complete understanding of the subject matter may be derived by referring to the detailed description and claims when considered in conjunction with the following figures, wherein like reference numbers refer to similar elements throughout the figures, which may be illustrated for simplicity and clarity and are not necessarily drawn to scale.

FIG. 1 depicts an exemplary embodiment of a patient monitoring system;

FIG. 2 is a flow diagram of an exemplary data transfer process suitable for use in the patient monitoring system of FIG. 1 in one or more exemplary embodiments;

FIGS. 3-5 depict a sequence of graphical user interface displays that may be presented in conjunction with the data transfer process of FIG. 2 in one or more exemplary embodiments;

FIG. 6 is a flow diagram of an exemplary event monitoring process suitable for use in the patient monitoring system of FIG. 1 in one or more exemplary embodiments;

FIGS. 7-18 depict various graphical user interface displays that may be presented on a client computing device in conjunction with the event monitoring process of FIG. 6 in one or more exemplary embodiments;

FIG. 19 depicts an embodiment of a computing device of a diabetes data management system suitable for use in connection with one or more of the patient monitoring system of FIG. 1 and the processes of FIGS. 2 and 6 in accordance with one or more embodiments;

FIG. 20 depicts an exemplary embodiment of an infusion system suitable for use in connection with one or more of the patient monitoring system of FIG. 1 and the processes of FIGS. 2 and 6 in accordance with one or more embodiments;

FIGS. 21-22 depict exemplary graphical user interface displays including event log indicia that may be presented on a display device associated with a computing device in accordance with one or more embodiments of the event monitoring process of FIG. 6;

FIG. 23 is a flow diagram of an exemplary display toggling process suitable for use in the patient monitoring system of FIG. 1 in one or more exemplary embodiments; and

FIGS. 24-25 depict exemplary graphical user interface displays including graphical representations of measurement data that may be presented in conjunction with the display toggling process of FIG. 23 in one or more exemplary embodiments.

### DETAILED DESCRIPTION

The following detailed description is merely illustrative in nature and is not intended to limit the embodiments of the subject matter or the application and uses of such embodiments. As used herein, the word “exemplary” means “serving as an example, instance, or illustration.” Any implementation described herein as exemplary is not necessarily to be construed as preferred or advantageous over other implementations. Furthermore, there is no intention to be bound by any expressed or implied theory presented in the preceding technical field, background, brief summary or the following detailed description.

Exemplary embodiments of the subject matter described herein are implemented in conjunction with medical devices, such as portable electronic medical devices. Although many different applications are possible, the following description may focus on embodiments that incorporate a glucose sensing arrangement for purposes of substantially continuous glucose monitoring. In one or more embodiments described herein, the monitoring device periodically and autonomously provides, to a remote device via an intermediary, measurement data that quantifies, characterizes, or otherwise correlates to the glucose level of the user. The remote device stores or otherwise maintains the measurement data to support subsequent analysis of the measurement data to for determining the manner in which an individual's therapy or lifestyle should be modified or otherwise adjusted to improve glucose control.

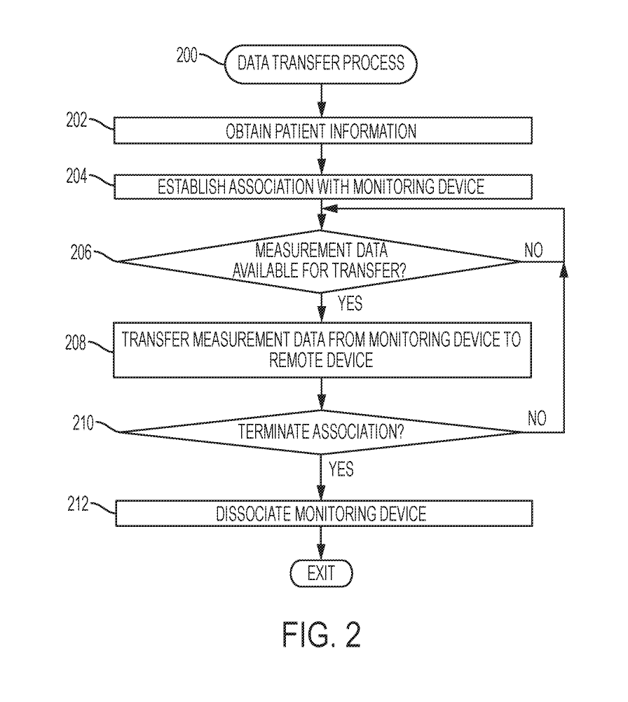

As described in greater detail below in the context of FIGS. 2-5, in exemplary embodiments, an intermediate device is paired with the monitoring device, and temporary communications sessions between the intermediate device and the monitoring device are utilized to autonomously upload recently obtained measurement data from the monitoring device to the remote device via the paired intermediate device without manual oversight or interaction. For example, when the monitoring device determines that recently obtained measurement data should be provided to the monitoring device, the monitoring device may provide an indication to the intermediate device to autonomously initiate establishment of communications sessions that are utilized to transmit the measurement data before being terminated. In this regard, the intermediate device provides an interface between a first communications network that the monitoring device is capable of communicating on and another communications network on which the remote device communicates. For example, the infusion device may communicate on a personal area network (PAN) or another short range communications network while the monitoring device communicates on the Internet, a cellular network, or the like. Thus, the measurement data may be effectively streamed from the monitoring device to the remote device via the intermediate device periodically and autonomously without requiring human intervention or a direct communications session between the monitoring device and the remote device.

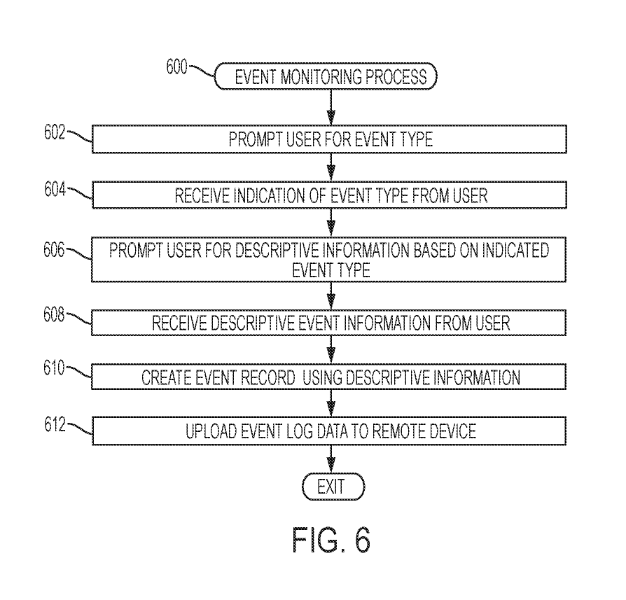

As described in greater detail below in the context of FIGS. 6-18, in exemplary embodiments, the intermediate device also supports a user manually logging or journaling events, activities, or other conditions or contextual information that may influence or impact the user's glucose levels The manually-entered event log data is also uploaded to the remote device by the intermediate device. In some embodiments, the event log data is uploaded substantially immediately upon creation or entry of information for a new event to be logged, however, in alternative embodiments, the event log data may be stored and maintained at the intermediate device for uploading subsequently in response to receiving an upload indication from the user or in concert with uploading measurement data from the monitoring device.

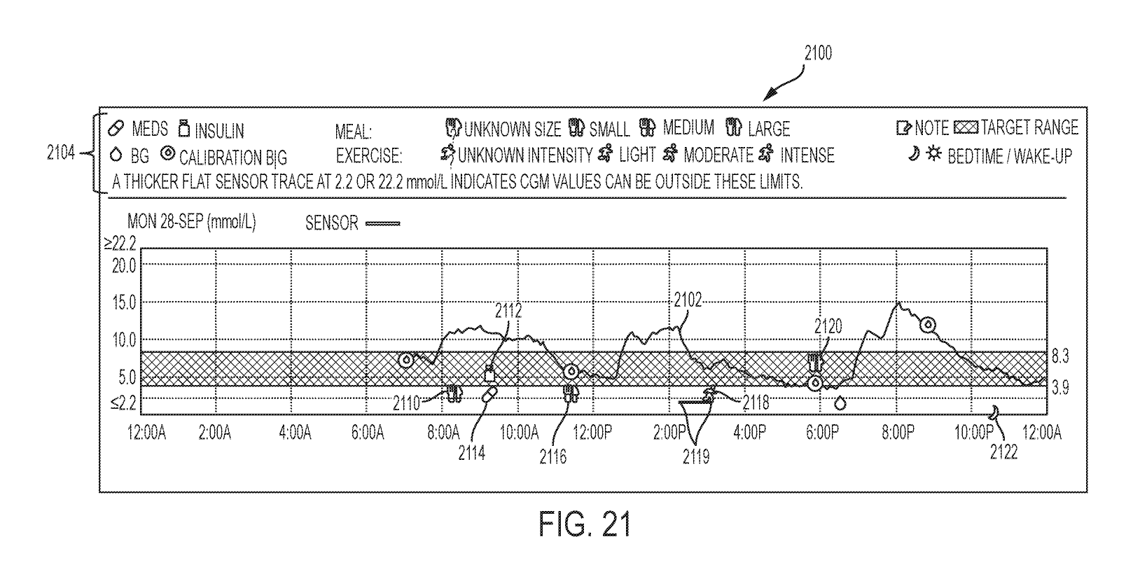

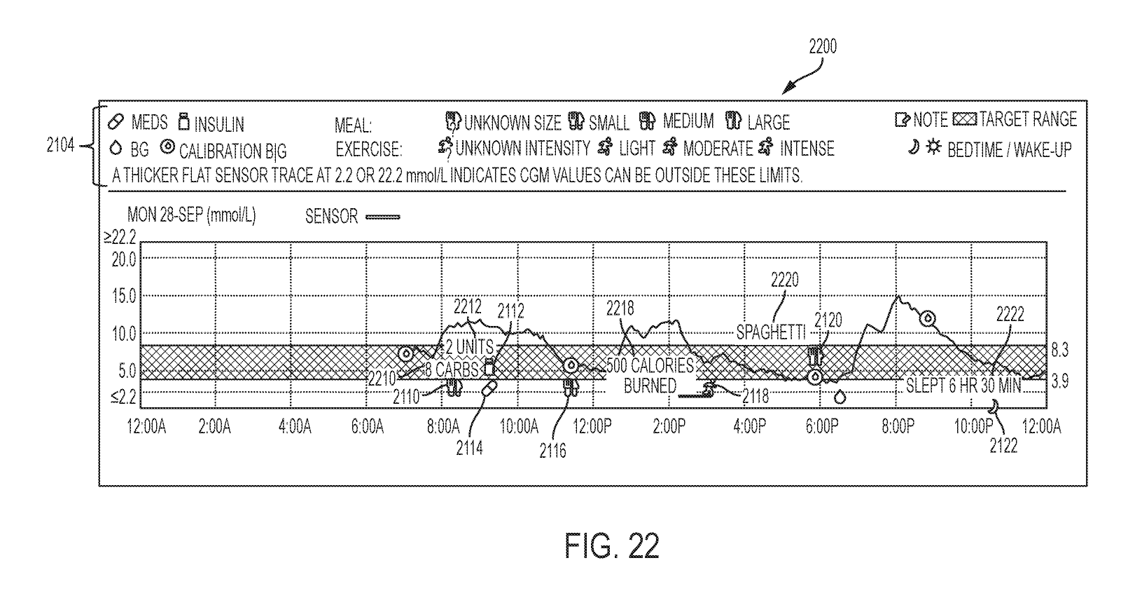

The uploaded measurement event log data may be analyzed at the remote device to support presenting information pertaining to the user's glycemic profile. For example, a snapshot graphical user interface (GUI) display may be presented on an electronic device coupled to the remote device, and the snapshot GUI display may include or otherwise provide graphical representations or other graphical indicia of the glucose measurement data over a snapshot time period, the events logged by the user during the snapshot period, and potentially various other aspects pertaining to the user's physiological condition. For example, the snapshot GUI display may include graphical representations of a diabetic patient's glucose levels along with other indicia or overlays pertaining to meals, boluses, medications, exercise, or other activities documented in the event log, as described in greater detail below in the context of FIGS. 21-22. One or more examples of a snapshot GUI display are provided in U.S. Patent Pub. No. 2017/0106144.

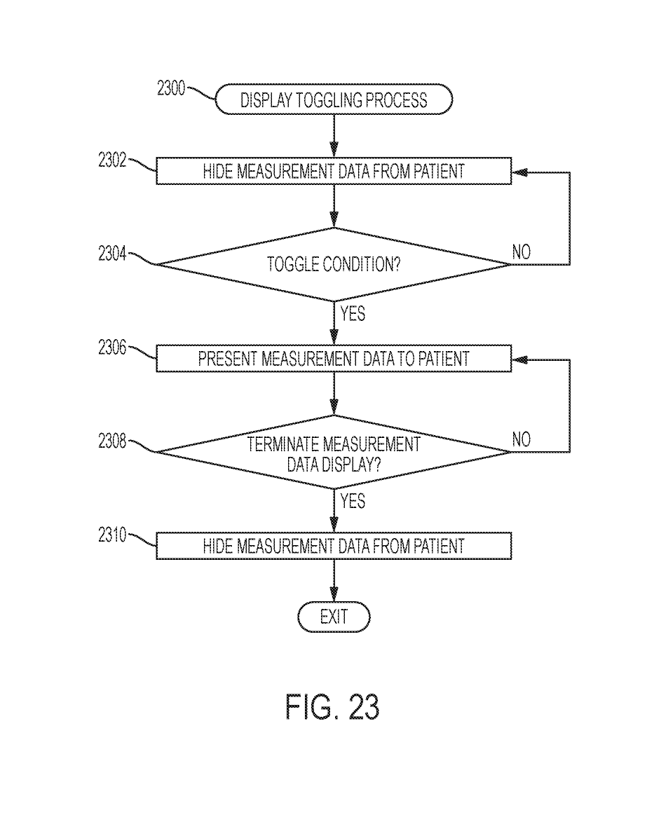

In exemplary embodiments, the measurement data from the monitoring device is normally hidden from or otherwise invisible to the patient or other user of the intermediate device. As described in greater detail below in the context of FIGS. 23-25, one or more toggling conditions may be detected during a monitoring period, and in response, a graphical user interface display on the intermediate device may be automatically toggled or switched from the normal or default display mode where the measurement data is not presented to a diagnostic display mode that includes a graphical representation of at least some of the measurement data. For example, in some embodiments, a chart or graph of the measurement data including the current or most recent measurement data and at least some of the patient's preceding measurement data may be presented. In other embodiments, the current or most recent sensor glucose measurement value may be presented to that the patient or user can monitor his or her glucose level essentially in real-time. In such embodiments, new or updated sensed glucose measurement values may be provided by the monitoring device to the intermediate device in a continual manner to facilitate real-time updates to the depicted measurement data as new measurement data becomes available. In one or more exemplary embodiments, the diagnostic display mode is temporary and the normal display mode is automatically transitioned to after an elapsed time in the diagnostic display mode exceeds a threshold duration or time or identification some other condition triggers a change in the display state.

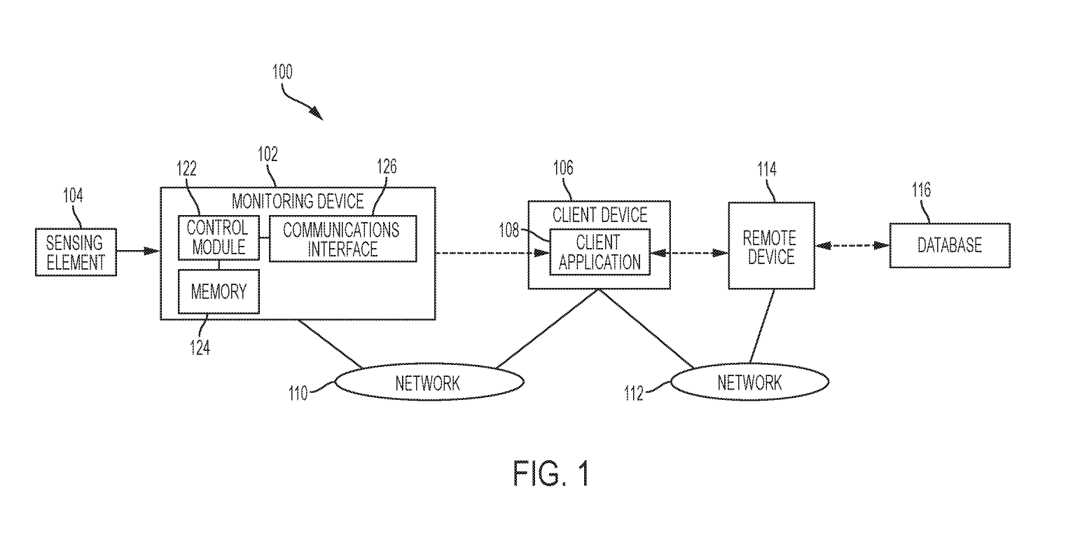

FIG. 1 depicts an exemplary embodiment of a patient monitoring system 100. The patient monitoring system 100 includes a monitoring device 102 that is communicatively coupled to a sensing element 104 that is inserted into the body of a patient or otherwise worn by the patient to obtain measurement data indicative of a physiological condition in the body of the patient, such as a sensed glucose level. The monitoring device 102 is communicatively coupled to a client device 106 via a communications network 110, with the client device 106 being communicatively coupled to a remote device 114 via another communications network 112. In this regard, the client device 106 functions as an intermediary for uploading or otherwise providing measurement data from the monitoring device 102 to the remote device 114.

In exemplary embodiments, the client device 106 is realized as a mobile phone, a smartphone, a tablet computer, or other similar mobile electronic device; however, in other embodiments, the client device 106 may be realized as any sort of electronic device capable of communicating with the monitoring device 102 via network 110, such as a laptop or notebook computer, a desktop computer, or the like. In exemplary embodiments, the network 110 is realized as a Bluetooth network, a ZigBee network, or another suitable personal area network. That said, in other embodiments, the network 110 could be realized as a wireless ad hoc network, a wireless local area network (WLAN), or local area network (LAN). The client device 106 includes or is coupled to a display device, such as a monitor, screen, or another conventional electronic display, capable of graphically presenting data and/or information pertaining to the physiological condition of the patient. The client device 106 also includes or is otherwise associated with a user input device, such as a keyboard, a mouse, a touchscreen, or the like, capable of receiving input data and/or other information from the user of the client device 106.

In exemplary embodiments, a user, such as the patient, the patient's doctor or another healthcare provider, or the like, manipulates the client device 106 to execute a client monitoring application 108 that supports communicating with the monitoring device 102 via the network 110. In this regard, the client application 108 supports establishing a communications session with the monitoring device 102 on the network 110 and receiving data and/or information from the monitoring device 102 via the communications session. The monitoring device 102 may similarly execute or otherwise implement a corresponding application or process that supports establishing the communications session with the client application 108. The client application 108 generally represents a software module or another feature that is generated or otherwise implemented by the client device 106 to support the processes described herein. Accordingly, the client device 106 generally includes a processing system and a data storage element (or memory) capable of storing programming instructions for execution by the processing system, that, when read and executed, cause processing system to create, generate, or otherwise facilitate the client application 108 and perform or otherwise support the processes, tasks, operations, and/or functions described herein. Depending on the embodiment, the processing system may be implemented using any suitable processing system and/or device, such as, for example, one or more processors, central processing units (CPUs), controllers, microprocessors, microcontrollers, processing cores and/or other hardware computing resources configured to support the operation of the processing system described herein. Similarly, the data storage element or memory may be realized as a random access memory (RAM), read only memory (ROM), flash memory, magnetic or optical mass storage, or any other suitable non-transitory short or long term data storage or other computer-readable media, and/or any suitable combination thereof.

In one or more embodiments, the client device 106 and the monitoring device 102 establish an association (or pairing) with one another over the network 110 to support subsequently establishing a point-to-point or peer-to-peer communications session between the monitoring device 102 and the client device 106 via the network 110. For example, in accordance with one embodiment, the network 110 is realized as a Bluetooth network, wherein the monitoring device 102 and the client device 106 are paired with one another (e.g., by obtaining and storing network identification information for one another) by performing a discovery procedure or another suitable pairing procedure. The pairing information obtained during the discovery procedure allows either of the monitoring device 102 or the client device 106 to initiate the establishment of a secure communications session via the network 110.

In one or more exemplary embodiments, the client application 108 is also configured to store or otherwise maintain an address and/or other identification information for the remote device 114 on the second network 112. In this regard, the second network 112 may be physically and/or logically distinct from the network 110, such as, for example, the Internet, a cellular network, a wide area network (WAN), or the like. The remote device 114 generally represents a server or other computing device configured to receive and analyze or otherwise monitor measurement data, event log data, and potentially other information obtained for the patient associated with the monitoring device 102 and generate one or more GUI displays (e.g., a snapshot GUI display) that may be presented on the remote device 114 or another electronic device (e.g., another instance of a client device 106 coupled to the remote device 114 via network 112). In exemplary embodiments, the remote device 114 is coupled to a database 116 configured to store or otherwise maintain data associated with individual patients. In practice, the remote device 114 may reside at a location that is physically distinct and/or separate from the monitoring device 102 and the client device 106, such as, for example, at a facility that is owned and/or operated by or otherwise affiliated with a manufacturer of the monitoring device 102. For purposes of explanation, but without limitation, the remote device 114 may alternatively be referred to herein as a server.

A user, such as the patient's doctor or another healthcare provider, may manipulate a client device to execute a client application (such as a web browser application), contact the server 114 via the network 112, and input or otherwise provide indication of the patient for which the user would like to review, analyze, or otherwise assess measurement data associated therewith. In response, the server 114 accesses the database 116 to retrieve or otherwise obtain measurement data, event log data, and potentially other information associated with the identified patient for the desired time period and generates one or more GUI displays (e.g., snapshot GUI display) that is presented on the display device associated with the client device via the client application executing thereon, thereby allowing the patient's doctor or another healthcare provider to review and analyze the patient's measurement data and event log data and make appropriate therapy modifications or lifestyle recommendations.

Still referring to FIG. 1, the sensing element 104 generally represents the component of the patient monitoring system 100 that is configured to generate, produce, or otherwise output one or more electrical signals indicative of a physiological condition that is sensed, measured, or otherwise quantified by the sensing element 104. In this regard, the physiological condition of a user influences a characteristic of the electrical signal output by the sensing element 104, such that the characteristic of the output signal corresponds to or is otherwise correlative to the physiological condition that the sensing element 104 is sensitive to. In exemplary embodiments, the sensing element 104 is realized as an interstitial glucose sensing element inserted at a location on the body of the patient that generates an output electrical signal having a current (or voltage) associated therewith that is correlative to the interstitial fluid glucose level that is sensed or otherwise measured in the body of the patient by the sensing element 104. In one embodiment, in addition to the glucose measurement signal, the sensing element 104 also outputs or otherwise provides an indication of a characteristic impedance associated with the sensing element 104. The characteristic impedance may be utilized to assess sensor performance (e.g., accuracy, sensitivity, or the like), remaining usage life, and the like.

The monitoring device 102 generally represents the component of the patient monitoring system 100 that is communicatively coupled to the output of the sensing element 104 to receive or otherwise obtain the measurement data samples from the sensing element 104 (e.g., the measured glucose and characteristic impedance values), store or otherwise maintain the measurement data samples, and upload or otherwise transmit the measurement data to the server 114 via the client device 106. It should be noted that although FIG. 1 depicts the monitoring device 102 and the sensing element 104 as separate components, in practice, the monitoring device 102 and the sensing element 104 may be integrated or otherwise combined to provide a unitary device that can be worn by the patient.

In exemplary embodiments, the monitoring device 102 includes a control module 122, a data storage element 124 (or memory), and a communications interface 126. The control module 122 generally represents the hardware, circuitry, logic, firmware and/or other component(s) of the monitoring device 102 that is coupled to the sensing element 104 to receive the electrical signals output by the sensing element 104 and perform or otherwise support various additional tasks, operations, functions and/or processes described herein. Depending on the embodiment, the control module 122 may be implemented or realized with a general purpose processor, a microprocessor, a controller, a microcontroller, a state machine, a content addressable memory, an application specific integrated circuit, a field programmable gate array, any suitable programmable logic device, discrete gate or transistor logic, discrete hardware components, or any combination thereof, designed to perform the functions described herein. In some embodiments, the control module 122 includes an analog-to-digital converter (ADC) or another similar sampling arrangement that samples or otherwise converts an output electrical signal received from the sensing element 104 into corresponding digital measurement data value. In other embodiments, the sensing element 104 may incorporate an ADC and output a digital measurement value.

In exemplary embodiments, the control module 122 stores or otherwise maintains glucose measurement values and characteristic impedance values obtained from the sensing element 104 in the memory 124 until subsequent transmission to the remote device 114. In exemplary embodiments, the memory 124 is realized as flash memory, however, in alternative embodiments, the memory 124 could be realized using any sort of RAM, ROM, registers, hard disks, removable disks, magnetic or optical mass storage, short or long term storage media, or any other non-transitory computer-readable medium.

The communications interface 126 generally represents the hardware, circuitry, logic, firmware and/or other components of the monitoring device 102 that are coupled to the control module 122 for outputting data and/or information from/to the monitoring device 102 to/from the client device 106. For example, the communications interface 126 may include or otherwise be coupled to one or more transceiver modules capable of supporting wireless communications between the monitoring device 102 and the client device 106. In exemplary embodiments, the communications interface 126 is realized as a Bluetooth transceiver or adapter configured to support Bluetooth Low Energy (BLE) communications.

It should be appreciated that FIG. 1 depicts a simplified representation of a patient monitoring system 100 for purposes of explanation and is not intended to limit the subject matter described herein in any way. For example, while FIG. 1 depicts a single sensing element 104 and monitoring device 102, in practice, embodiments of the patient monitoring system 100 may include multiple different sensing arrangements and/or monitoring devices, which may be configured to sense, measure, or otherwise quantify any number of conditions or characteristics. In this regard, multiple instances of a glucose sensing element 104 and/or monitoring devices 102 may be deployed for redundancy or other purposes (e.g., averaging or other statistical operations), or to measure different physiological conditions of the patient, such as, for example, the patient's heart rate, oxygen levels, or the like.

FIG. 2 depicts an exemplary data transfer process 200 suitable for implementation in a patient monitoring system to transfer measurement data from a monitoring device to a remote device. The various tasks performed in connection with the data transfer process 200 may be performed by hardware, firmware, software executed by processing circuitry, or any combination thereof. For illustrative purposes, the following description refers to elements mentioned above in connection with FIG. 1. In practice, portions of the data transfer process 200 may be performed by different elements of the patient monitoring system 100, such as, for example, the monitoring device 102, the client device 106, the client application 108, and/or the remote device 114. It should be appreciated that the data transfer process 200 may include any number of additional or alternative tasks, the tasks need not be performed in the illustrated order and/or the tasks may be performed concurrently, and/or the data transfer process 200 may be incorporated into a more comprehensive procedure or process having additional functionality not described in detail herein. Moreover, one or more of the tasks shown and described in the context of FIG. 2 could be omitted from a practical embodiment of the data transfer process 200 as long as the intended overall functionality remains intact.

The illustrated data transfer process 200 begins by receiving or otherwise obtaining identifying information associated with the patient to be monitored using a monitoring device (task 202). In exemplary embodiments, the patient or other user manipulates a user interface of the client device 106 to input or otherwise provide information associated with the patient to the client application 108, and thereby establish an association between the instance of the client application 108 on the client device 106 and a record associated with that patient in the database 116. In this regard, for a new patient to be monitored, the patient or other user manipulates GUI elements (e.g., text boxes, check boxes, list boxes, combo boxes, and the like) provided by the client application 108 to input or otherwise provide patient information, such as, for example, the patient's name, the patient's date of birth, type of diabetes or other physiological condition which the patient exhibits or for which the patient is being monitored, the type(s) of therapy regimen(s) the patient is engaged in, identification of the patient's physician or healthcare provider, and the like. Once the patient information has been entered, a button or similar GUI element of the client application 108 may be selected to initiate transmission of the input patient information to the server 114 via the network 112 to create a record for the patient in the database 116. In this regard, a patient monitoring record in the database 116 may maintain an association between the patient information, identification information for the client device 106 and/or the instance of the client application 108, and the like. In embodiments where a record for the patient already exists in the database 116, the patient or other user may input or otherwise patient identification information or other login information or credentials to establish an association with the current client device 106 and/or the current instance of the client application 108 (e.g., by updating the patient record in the database 116 to include identification information for the client device 106 and/or the instance of the client application 108).

The data transfer process 200 continues by establishing an association between the client application and the monitoring device (task 204). In this regard, in exemplary embodiments, the client application 108 receives or otherwise obtains information that may be utilized to identify the monitoring device 102 on the network 110 and establish communications sessions with the monitoring device 102. In some embodiments, the client device 106 and the monitoring device 102 are configured to support a discovery procedure or similar pairing procedure in accordance with a communications protocol associated with the network 110, during which the client application 108 obtains and stores network identification information and potentially other information associated with the monitoring device 102 that allows the client application 108 to initiate the establishment of a secure communications session with the monitoring device 102 via the network 110. For example, the client device 106 and the monitoring device 102 may communicate exchange identification information with one another (e.g., providing a network address or other identification information on the network 110 along with any unique identifiers associated with the respective device 102, 106 and receiving the same from the other device 102, 106) and store the received information from the other device 102, 106 to thereby establish an association between devices 102, 106 that may be utilized for communications over the network 110. In some embodiments, the client application 108 also transmits or otherwise provides the identification information associated with the monitoring device 102 to the server 114 via the network 112, which, in turn may update the patient record in the database 116 to maintain an association between the patient and the monitoring device 102.

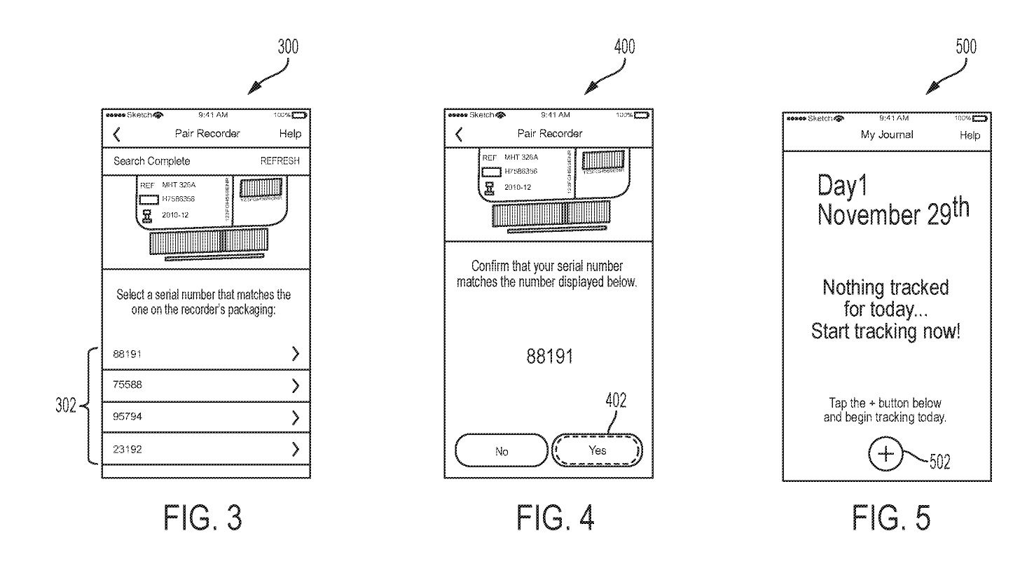

As described in greater detail below in the context of FIGS. 3-5, in some embodiments, the patient or other user manipulates one or more GUI elements provided by the client application 108 to provide identification of the monitoring device 102 to be paired with the client application 108, which, in turn may be utilized by the client application 108 to establish an association with the monitoring device 102. For example, the client application 108 may perform a scan procedure or similar process to discover potential monitoring devices within communications range of the client device 106 and provide a GUI display including a listing of the detected monitoring devices within range of the client device 106. In response to selection or indication of a particular monitoring device from the list, the client application 108 may proceed with a pairing procedure to establish an association with the selected monitoring device 102.

Still referring to FIG. 2, the data transfer process 200 continues by identifying or otherwise determining whether there is measurement data available for uploading to the remote device (task 206). In one or more exemplary embodiments, the client application 108 periodically polls the monitoring device 102 for an indication that measurement data is ready for uploading. In this regard, to conserve energy, the monitoring device 102 may store measurement data samples yet to be uploaded in memory 124, and then once the amount of stored measurement data samples exceeds a threshold, the monitoring device 102 provides an indication to the client application 108 that measurement data at the monitoring device 102 is ready for uploading. In one or more embodiments, the monitoring device 102 may transmit or otherwise provide a data ready indication to the client device 106 once at least six hours' worth of recent measurement data has been stored in the memory 124. In various alternative embodiments, the client application 108 may also poll or otherwise request recent measurement data from the monitoring device 102 whenever the client application 108 becomes active or is in the foreground on the client device 106, or when the client application 108 is transferring event log data or other information to the server 114.

For example, in one or more embodiments, the monitoring device 102 is realized as a continuous glucose monitor (CGM) that obtains sensed glucose measurement values at one minute intervals. To conserve battery life, rather than continually transferring measurement values, firmware supported by the control module 122 at the monitoring device 102 may attempt to manage when the client application 108 requests measurement data by advertising available measurement data during the periodic BLE advertising period once at least 6 hours of yet to be uploaded measurement data is available at the monitoring device 102. For example, a data ready flag in a BLE advertisement packet may be set to true to indicate at least 6 hours' worth of measurement data samples is available at the monitoring device 102. In this regard, the client application 108 may restrict, limit, or prevent establishing communications sessions with the monitoring device 102 when the data ready flag is set to false. When the data ready flag a BLE advertisement packet is set to true, the client application 108 may initiate a BLE communications session with the monitoring device 102 to request and receive the recent measurement data from the monitoring device 102, which, in turn, may be automatically uploaded to the server 114. In this manner, measurement data may be uploaded from the monitoring device 102 to the server 114 via the client device 106 in the background without user interaction and with the measurement data being maintained hidden from the user.

When measurement data is available, the data transfer process 200 continues by uploading or otherwise transferring measurement data from the monitoring device to the remote device via the client device (task 208). In this regard, the client application 108 utilizes the stored identification information for the monitoring device 102 to establish a point-to-point communications session over the network 110 and retrieve the stored measurement data (e.g., six hours of sensor glucose measurement values and characteristic impedance values) from the monitoring device 102 via the network 110. The client application 108 establishes a secure communications session over the network 112 with the server 114 and then transmits or otherwise uploads the retrieved measurement data to the server 114 via the network 112. In exemplary embodiments, the server 114 stores the measurement data for the patient in the database 116 in association with the patient's record in the database 116. After the measurement data is uploaded, the monitoring device 102 may tag or otherwise mark the measurement data samples in memory 124 as having been uploaded, or in alternative embodiments, the monitoring device 102 may delete uploaded measurement data from the memory 124 to ensure sufficient space exists in memory 124 for subsequent measurement data.

In exemplary embodiments, the data transfer process 200 determines whether to terminate the association with the monitoring device (task 210). For example, the client application 108 may support monitoring the patient for a finite duration (e.g., a 7 day study), and once the client application 108 determines that measurement data corresponding to that monitoring period duration has been obtained and uploaded from the monitoring device 102 to the server 114, the client application 108 may determine that the association with the monitoring device 102 can be terminated. In this regard, when the monitoring period duration has not elapsed or uploading additional measurement data is still desired, the loop defined by tasks 206, 208 and 210 repeats until sufficient measurement data has been uploaded to the remote device.

After determining the association with the monitoring device can be terminated, the data transfer process 200 dissociates the monitoring device (task 212). For example, once 7 days' worth of measurement data has been uploaded, the client application 108 may autonomously transmit or otherwise provide an indication to the monitoring device 102 that the measurement data has been uploaded to the server 114 and that the devices 102, 106 can be dissociated. In one or more embodiments, the client application 108 provides a study completed status indication to the monitoring device 102. In response to the study completed status indication, the firmware at the control module 122 causes the monitoring device 102 to establish a communications session with the client device 106 to upload or otherwise transmit diagnostic data to the client application 108. After uploading diagnostic data, the firmware at the control module 122 may be configured to automatically delete any measurement data, diagnostic data, or other patient-specific information from the memory 124 along with any identification information or pairing information corresponding to the client device 106, to thereby terminate communications with the client device 106.

FIGS. 3-5 depict an exemplary sequence of GUI displays that may be presented on the client device 106 by the client application 108 in connection with the data transfer process 200 to pair a monitoring device 102 with the client device 106. In response to a user manipulating a GUI element of the client application 108 to initiate a scan procedure, the client application 108 may cause the client device 106 to scan for monitoring devices within communications range, obtain identifying information for the monitoring devices within communications range, and then generate a GUI display 300 including a list 302 of the detected monitoring devices. The user identifies the monitoring device 102 that the patient is or will be wearing and selects that monitoring device 102 from within the list 300. In response to selection of the desired monitoring device 102, the client application 108 provides a confirmation GUI display 400 that includes a button or similar selectable GUI element 402 for confirming the selected monitoring device from the list 302 is the monitoring device 102 the patient will be wearing. In response to selection of the confirmation button 402, the client application 108 initiates a pairing procedure using the identification information associated with the selected monitoring device to establish an association with the monitoring device 102 on the network 110, as described above.

After pairing the selected monitoring device 102 with the client device 106, the client application 108 generates or otherwise provides a home screen GUI display 500 associated with the client application 108. The home screen GUI display 500 may include notifications or other information pertaining to the monitoring period, such as, for example, the current date and current day of the study, an event log, and other information pertaining monitoring period. In this regard, in exemplary embodiments, the home screen GUI display 500 includes a selectable GUI element 502 that allows the patient to input or otherwise provide descriptive information pertaining to the patient's activities, meals, or other lifestyle events during the monitoring period.

FIG. 6 depicts an exemplary event monitoring process 600 suitable for implementation in patient monitoring system to log or otherwise track data pertaining to events experienced by a patient during a monitoring period that may influence the patient's physiological condition being monitored by a monitoring device. In this regard, the event monitoring process 600 may be performed concurrently to or in concert with the data transfer process 200 of FIG. 2. The various tasks performed in connection with the event monitoring process 600 may be performed by hardware, firmware, software executed by processing circuitry, or any combination thereof. For purposes of explanation, the event monitoring process 600 may be described herein as primarily being performed or supported by the client application 108 at the client device 106. It should be appreciated that the event monitoring process 600 may include any number of additional or alternative tasks, the tasks need not be performed in the illustrated order and/or the tasks may be performed concurrently, and/or the event monitoring process 600 may be incorporated into a more comprehensive procedure or process having additional functionality not described in detail herein. Moreover, one or more of the tasks shown and described in the context of FIG. 6 could be omitted from a practical embodiment of the event monitoring process 600 as long as the intended overall functionality remains intact.

In exemplary embodiments, the event monitoring process 600 is initiated or otherwise performed in response to a user indicating a desire to document, journal, or otherwise log an event during the monitoring period by selecting a GUI element (e.g., GUI element 502) on a GUI display provided by the client application 108. The event monitoring process 600 prompts the user of the client device to select, input or otherwise provide the event type that the user would like to log and receives indication of the event type from the user (tasks 602, 604). In an exemplary embodiment, the client application 108 generates a plurality of selectable GUI elements corresponding to the various types of lifestyle events that are likely to influence the patient's physiological condition, such as, for example, exercise, medication, meals, sleep, and the like. In other embodiments, the client application 108 may generate or provide a list box, a text box, or other suitable GUI element for receiving an input event type.

In response to receiving indication of the type of lifestyle event to be logged, the event monitoring process 600 continues by prompting the user for descriptive information characterizing the lifestyle event in a manner that is influenced by the indicated event type (task 606). In this regard, as described in greater detail below, depending on the particular event type indicated by the user, the client application 108 may generate or otherwise provide GUI elements that prompt the user to input different types and/or amounts of descriptive information or attributes characterizing different aspects of the type of lifestyle event that are likely to influence the patient's physiological condition.

The event monitoring process 600 continues by receiving the descriptive information characterizing the event from the user and creating an event record that maintains an association between the event type and the descriptive information (tasks 608, 610). In this regard, the client application 108 may maintain an event log associated with the patient consisting of event records created by the patient in a memory of the client device 106. For example, the client application 108 may assign an event identifier to the event and create a record in memory at the client device 106 that maintains an association between the event identifier, its associated event type, and the descriptive information input by the user. In some embodiments, one or more input values for an attribute input by the user may be utilized to generate the event identifier, with the remaining input descriptive information and/or attribute values stored as fields of metadata associated with the event record.

After creating the event record, the event monitoring process 600 continues by uploading or otherwise transmitting the event record to the remote device for storage in association with the patient (task 612). In this regard, the server 114 may maintain an event log associated with the patient in the database 116, with the event log maintaining the individual event records in association with the monitoring period for the patient. In some embodiments, when the client device 106 is capable of communicating on the network 112, the client application 108 automatically establishes a communications session with the server 114 over the network 112 and automatically uploads the event record upon creation. In this manner, the event log maintained in the database 116 may be automatically synced with the event log at the client device 106 when the client device 106 is capable of communicating with the server 114. In yet other embodiments, the client application 108 may establish the communications session and upload the event record in response to receiving an indication from the user. For example, the client application 108 may generate or otherwise provide a GUI element that is selectable by the user to initiate uploading of any event records stored locally at the client device 106 that have not yet been uploaded to the server 114. In various embodiments, the client application 108 may store the event record and subsequently prompt the user to upload event records, for example, on a periodic basis (e.g., daily), in response to the number of yet to be uploaded event records at the client device 106 exceeding a threshold number, in response to identifying measurement data ready for uploading at the monitoring device 102 (e.g., in response to a data ready flag value of true), or in response to some other event.

After the monitoring period, the server 114 may utilize the event log data associated with the patient uploaded in connection with the event monitoring process 600 in conjunction with the measurement data associated with the patient uploaded in connection with the data transfer process 200 to generate a snapshot GUI display or other GUI display representative of the patient's physiological condition during the monitoring period. For example, the server 114 may generate a graph or similar graphical representation of the patient's sensed glucose measurement values during the monitoring period with overlaid graphical representations or indicia of one or more event records of the patient's event log (e.g., meal indicators, exercise indicators, or the like). In this regard, descriptive information or metadata input by the patient for an event may be utilized by the server 114 to temporally relate or otherwise associate the event with a subset of the glucose measurement data and graphically depict the relationship on a client device.

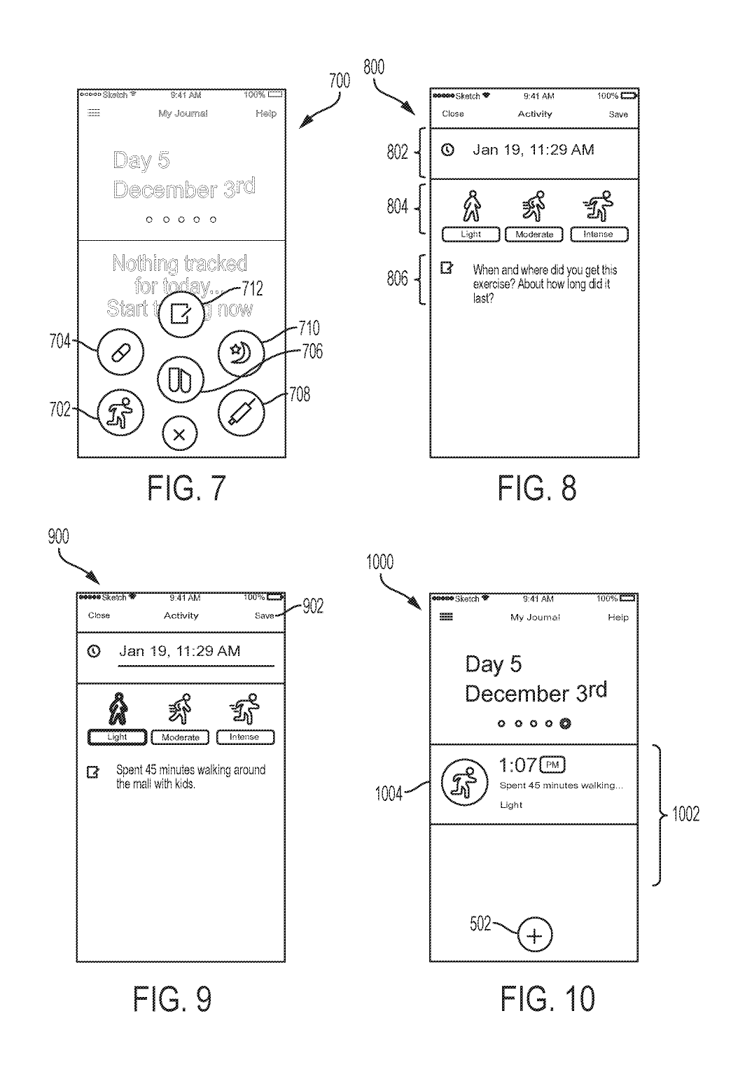

FIG. 7 depicts an exemplary event type selection GUI display 700 that may be presented by the client application 108 on the client device 106 in connection with the event monitoring process 600 of FIG. 6. In this regard, the event type selection GUI display 700 may be presented by the client application 108 on the client device 106 in response to selection of the add event button 502 on the home screen GUI display 500. The event type selection GUI display 700 includes a plurality of selectable GUI elements 702, 704, 706, 708, 710 for indication of the type of lifestyle event the user would like to add to the event log, such as, for example, an exercise button 702, a medication button 704, a meal button 706, an injection (or bolus) button 708, and a sleep button 710. Additionally, the event type selection GUI display 700 includes a button 712 for adding notes or other descriptive information pertaining to lifestyle activities or events that are not classifiable into one of the event types corresponding to buttons 702, 704, 706, 708, 710. In response to selection of a GUI element 702, 704, 706, 708, 710, 712, the client application 108 updates the GUI display on the client device 106 to provide GUI elements that prompt the user to input attribute values or other descriptive information or metadata corresponding to the selected type of event to be added to the event log.

For example, referring now to FIGS. 8-9, in response to selection of the exercise button 702, the client application 108 provides an exercise description GUI display 800 on the client device 106 that includes a first region 802 including one or more GUI elements for inputting the date and time of the exercise, a second region 804 including selectable GUI elements for characterizing the intensity of the exercise, and a third region 806 including a text box or similar GUI element for adding notes or other descriptive information pertaining to the exercise event being logged. As the user of the client device 106 manipulates the GUI elements to input descriptive information pertaining to the exercise event, the client application 108 generates an updated exercise description GUI display 900 that reflects the descriptive information and GUI selections received from the user. Once the user has finished characterizing the exercise event, the user selects a button or similar GUI element 902 to create an event record corresponding to the exercise event that maintains an association between the date and time of the exercise, the selected exercise intensity, and the other descriptive information provided by the user.

Referring to FIG. 10, after creating the exercise event record, the client application 108 generates an updated home screen GUI display 1000 having an event log region 1002 including graphical representation 1004 of the exercise event record. In this regard, the event log region 1002 may include a list or feed of event records created by the client application 108. In one or more embodiments, the event record depictions within the event log region 1002 are selectable by the user to review or edit the selected event record. For example, the user may select the exercise event record 1004 within the event log region 1002 to cause the client application 108 to revert to the updated exercise description GUI display 900 to review and/or edit the descriptive information associated with the exercise event record.

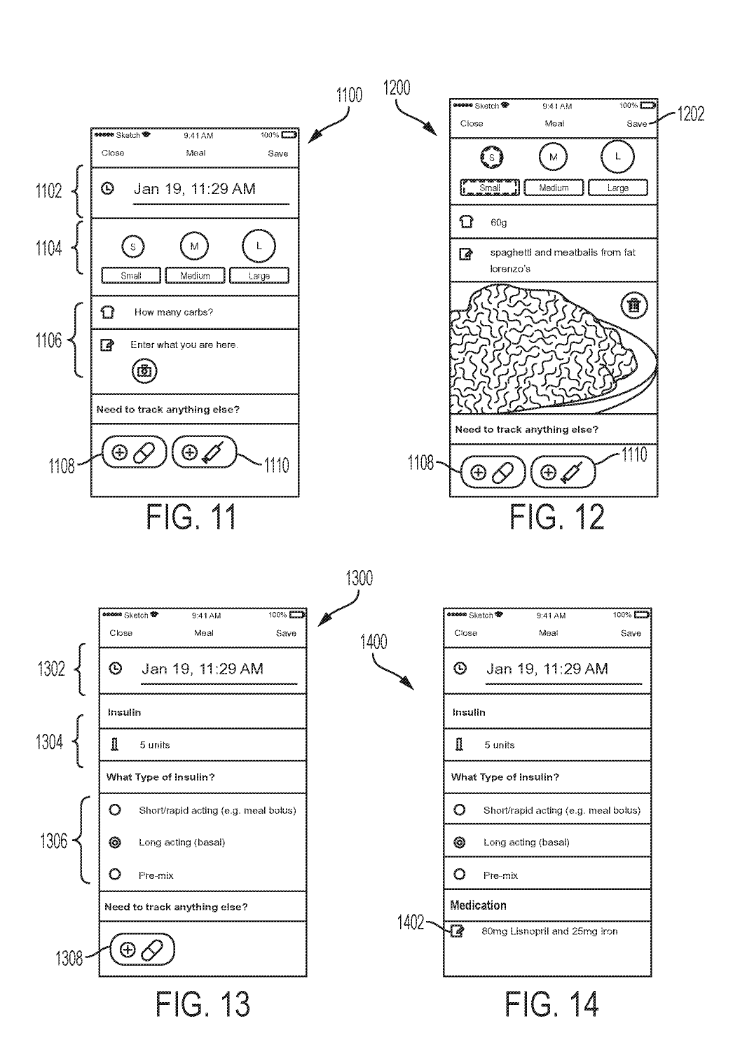

FIGS. 11-12 depict a sequence of GUI displays that may be presented by the client application 108 in response to selection of the meal button 706. In a similar manner as described above, the client application 108 provides a meal description GUI display 1100 that includes a first region 1102 including one or more GUI elements for inputting the date and time of the meal, a second region 1104 including selectable GUI elements for characterizing the size of the meal, and a third region 1106 including text boxes or similar GUI elements for inputting the number of carbohydrates associated with the meal and adding notes or other descriptive information pertaining to the meal. In the illustrated embodiment, the meal description region 1106 also includes a button or similar GUI element for associating a photograph with the meal, for example, by enabling a camera of the client device 106 to capture a photograph or associating an image stored at the client device 106. The updated meal description GUI display 1200 in FIG. 12 reflects the meal attributes input by the user and the photograph associated with the meal by the user. Once the user has finished characterizing the exercise event, the user may select a button or similar GUI element 1202 to create an event record corresponding to the meal event that maintains an association between the date and time of the meal, the selected meal size, the number of carbohydrates associated with the meal, and the other descriptive information provided by the user. The illustrated meal description GUI display 1100 also includes selectable GUI elements 1108, 1110 for inputting descriptive information pertaining to additional events that are likely to be contemporaneous or otherwise related to a meal event, such as administration of an oral medication or a bolus of insulin.

FIG. 13 depicts a bolus description GUI display 1300 that may be presented by the client application 108 on the client device 106 in response to selection of the bolus button 708 on the event type selection GUI display 700 or the bolus button 1110 on a meal description GUI display 1100, 1200. The bolus description GUI display 1300 includes a first region 1302 including one or more GUI elements for inputting the date and time of the bolus, a second region 1304 including a text box, drop-down menu, or other GUI element for inputting the amount of units of insulin associated with the bolus, and a third region 1306 including radio buttons or similar GUI elements for selecting the type of insulin administered. The illustrated bolus description GUI display 1300 also includes a medication button 1308 to create or add a medication event record to the event log, for example, for patient's undergoing combination therapy. As illustrated in FIG. 14, in response to selection of the medication button 1308, the client application 108 may provide an updated bolus description GUI display 1400 that includes a medication region 1402 with GUI elements for inputting descriptive information pertaining to a medication event concurrent to the bolus event.

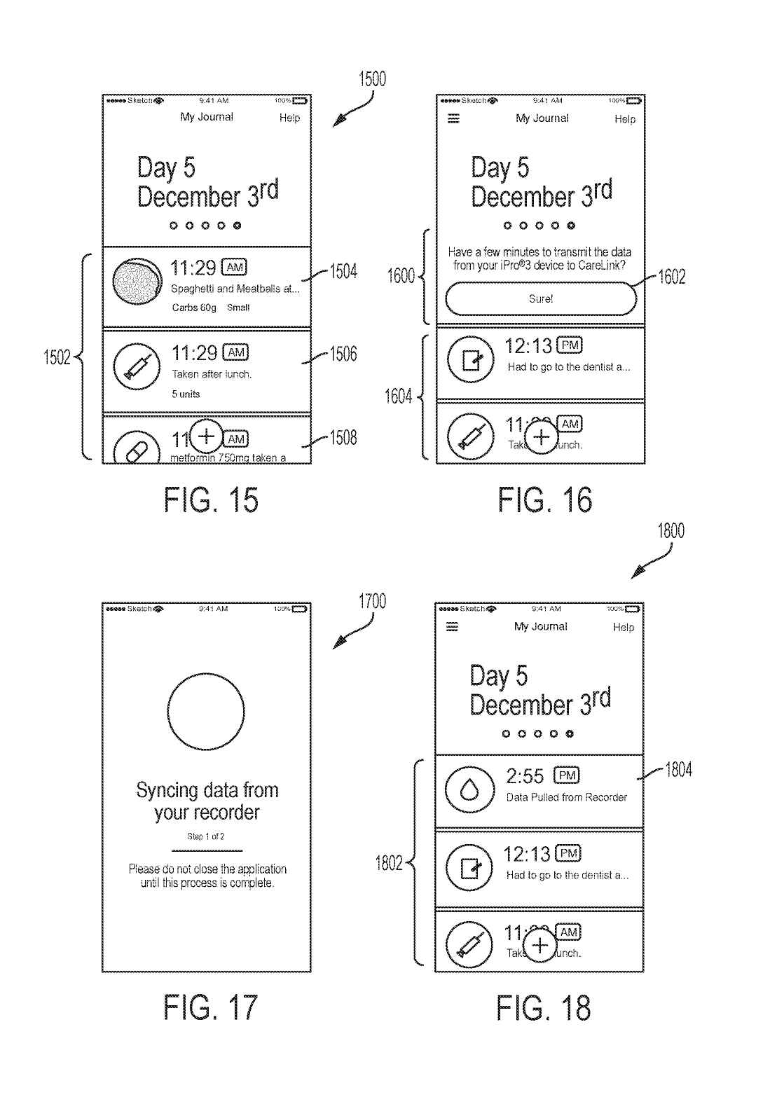

FIG. 15 depicts an updated home screen GUI display 1500 with an event log region 1502 that includes a list or feed of event records created using the client application 108. For example, the illustrated event log region 1502 includes a graphical representation 1504 of a meal event record corresponding to FIGS. 11-12, a graphical representation 1506 of a bolus event record contemporaneous to the meal event record corresponding to FIG. 13, and a graphical representation 1508 of a medication event record contemporaneous to the meal and bolus event records. In one or more exemplary embodiments, the log or feed of event records is scrollable, and the event records in the event log region 1502 are presented in reverse chronological order.

Referring now to FIGS. 16-18, in embodiments when the event records are not automatically uploaded to the server 114 upon creation (e.g., when connectivity to the network 112 is unavailable, disabled, or the like), the client application 108 may prompt the user to upload the event log data to the server 114, for example, on a periodic basis, in response to the client device 106 achieving connectivity to the network 112, in response to detecting available measurement data at the monitoring device 102, or the like. In this regard, the client application 108 may prompt the user by generating a region 1600 within the home screen GUI display above the event log region 1604 that includes text prompting the user to upload the event log data and a button or similar GUI element 1602 that is selectable to initiate uploading of the event records stored at the client device 106 to the server 114.

In response to selection of the button 1602, the client application 108 presents a data transfer notification GUI display 1700 on the client device 106 to notify the user of an ongoing attempt to transfer event log data to the server 114. The client application 108 concurrently establishes a secure communications session with the server 114 over the network 112 and uploads event log data stored at the client device 106 to the server 114 for storage in the database 116. In exemplary embodiments, the client application 108 identifies the event records at the client device 106 that have not been uploaded to the server 114 (e.g., the event records created since the preceding upload) and transmits only those event records to the server 114. Additionally, when the data ready flag broadcast by the monitoring device 102 is set to true, the client application 108 may establish a communications session with the monitoring device 102 to retrieve new measurement data and upload the new measurement data to the server 114 during the same communications session with the event log data. That said, in other embodiments, the client application 108 may automatically upload measurement data in the background and independently of the event log data.

Referring now to FIG. 18, in exemplary embodiments, after uploading the event log data, the client application 108 generates an updated home screen GUI display 1800 that includes a graphical indication 1804 of the event log data being uploaded to the server 114 within the event log region 1802. In this regard, the upload indication 1804 may be presented at the top of an event log feed ordered in reverse chronological order.

Diabetes Data Management System Overview

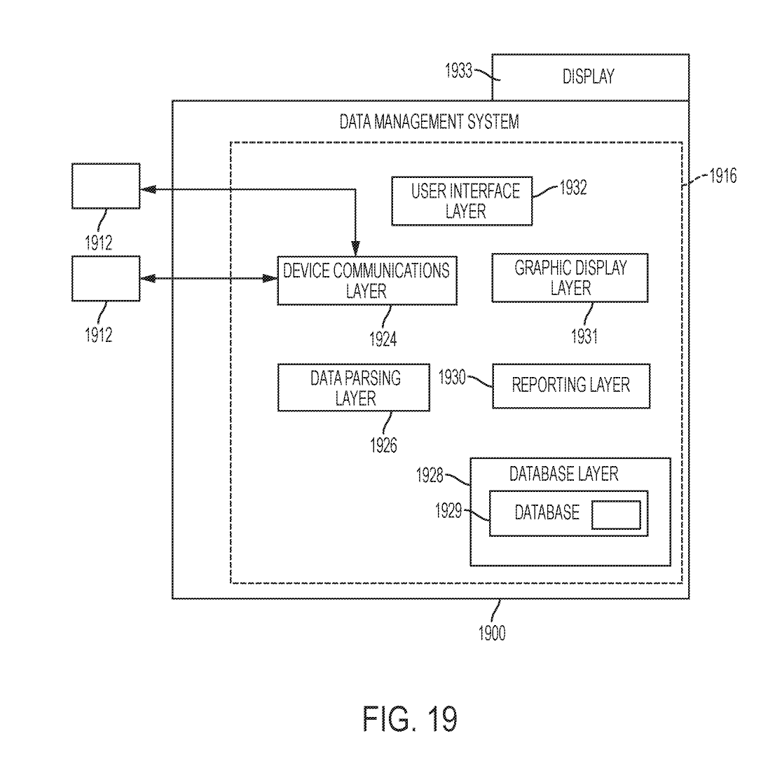

As described above, the uploaded event log data and measurement data may be utilized to present a snapshot GUI display including graphical representations of the measurement data over the monitoring period (or a subset thereof) and the events occurring during that time frame. In this regard, FIG. 19 illustrates a computing device 1900 including a display 1933 suitable for presenting a snapshot GUI display as part of a diabetes data management system in conjunction with the data transfer process 200 and the event monitoring process 600 described above. The diabetes data management system (DDMS) may be referred to as the Medtronic MiniMed CARELINK™ system or as a medical data management system (MDMS) in some embodiments. The DDMS may be housed on a server or a plurality of servers which a user or a health care professional may access via a communications network via the Internet or the World Wide Web. Some models of the DDMS, which is described as an MDMS, are described in U.S. Patent Application Publication Nos. 2006/0031094 and 2013/0338630, which is herein incorporated by reference in their entirety.

While description of embodiments are made in regard to monitoring medical or biological conditions for subjects having diabetes, the systems and processes herein are applicable to monitoring medical or biological conditions for cardiac subjects, cancer subjects, HIV subjects, subjects with other disease, infection, or controllable conditions, or various combinations thereof.

In embodiments of the invention, the DDMS may be installed in a computing device in a health care provider's office, such as a doctor's office, a nurse's office, a clinic, an emergency room, an urgent care office. Health care providers may be reluctant to utilize a system where their confidential patient data is to be stored in a computing device such as a server on the Internet.

The DDMS may be installed on a computing device 1900. The computing device 1900 may be coupled to a display 1933. In some embodiments, the computing device 1900 may be in a physical device separate from the display (such as in a personal computer, a mini-computer, etc.) In some embodiments, the computing device 1900 may be in a single physical enclosure or device with the display 1933 such as a laptop where the display 1933 is integrated into the computing device. In embodiments of the invention, the computing device 1900 hosting the DDMS may be, but is not limited to, a desktop computer, a laptop computer, a server, a network computer, a personal digital assistant (PDA), a portable telephone including computer functions, a pager with a large visible display, an insulin pump including a display, a glucose sensor including a display, a glucose meter including a display, and/or a combination insulin pump/glucose sensor having a display. The computing device may also be an insulin pump coupled to a display, a glucose meter coupled to a display, or a glucose sensor coupled to a display. The computing device 1900 may also be a server located on the Internet that is accessible via a browser installed on a laptop computer, desktop computer, a network computer, or a PDA. The computing device 1900 may also be a server located in a doctor's office that is accessible via a browser installed on a portable computing device, e.g., laptop, PDA, network computer, portable phone, which has wireless capabilities and can communicate via one of the wireless communication protocols such as Bluetooth and IEEE 802.11 protocols.

In the embodiment shown in FIG. 19, the data management system 1916 comprises a group of interrelated software modules or layers that specialize in different tasks. The system software includes a device communication layer 1924, a data parsing layer 1926, a database layer 1928, database storage devices 1929, a reporting layer 1930, a graph display layer 1931, and a user interface layer 1932. The diabetes data management system may communicate with a plurality of subject support devices 1912, two of which are illustrated in FIG. 19. Although the different reference numerals refer to a number of layers, (e.g., a device communication layer, a data parsing layer, a database layer), each layer may include a single software module or a plurality of software modules. For example, the device communications layer 1924 may include a number of interacting software modules, libraries, etc. In embodiments of the invention, the data management system 1916 may be installed onto a non-volatile storage area (memory such as flash memory, hard disk, removable hard, DVD-RW, CD-RW) of the computing device 1900. If the data management system 1916 is selected or initiated, the system 1916 may be loaded into a volatile storage (memory such as DRAM, SRAM, RAM, DDRAM) for execution.

The device communication layer 1924 is responsible for interfacing with at least one, and, in further embodiments, to a plurality of different types of subject support devices 1912, such as, for example, blood glucose meters, glucose sensors/monitors, or an infusion pump. In one embodiment, the device communication layer 1924 may be configured to communicate with a single type of subject support device 1912. However, in more comprehensive embodiments, the device communication layer 1924 is configured to communicate with multiple different types of subject support devices 1912, such as devices made from multiple different manufacturers, multiple different models from a particular manufacturer and/or multiple different devices that provide different functions (such as infusion functions, sensing functions, metering functions, communication functions, user interface functions, or combinations thereof). By providing an ability to interface with multiple different types of subject support devices 1912, the diabetes data management system 1916 may collect data from a significantly greater number of discrete sources. Such embodiments may provide expanded and improved data analysis capabilities by including a greater number of subjects and groups of subjects in statistical or other forms of analysis that can benefit from larger amounts of sample data and/or greater diversity in sample data, and, thereby, improve capabilities of determining appropriate treatment parameters, diagnostics, or the like.

The device communication layer 1924 allows the DDMS 1916 to receive information from and transmit information to or from each subject support device 1912 in the system 1916. Depending upon the embodiment and context of use, the type of information that may be communicated between the system 1916 and device 1912 may include, but is not limited to, data, programs, updated software, education materials, warning messages, notifications, device settings, therapy parameters, or the like. The device communication layer 1924 may include suitable routines for detecting the type of subject support device 1912 in communication with the system 1916 and implementing appropriate communication protocols for that type of device 1912. Alternatively or in addition, the subject support device 1912 may communicate information in packets or other data arrangements, where the communication includes a preamble or other portion that includes device identification information for identifying the type of the subject support device. Alternatively, or in addition, the subject support device 1912 may include suitable user-operable interfaces for allowing a user to enter information (e.g., by selecting an optional icon or text or other device identifier) that corresponds to the type of subject support device used by that user. Such information may be communicated to the system 1916, through a network connection. In yet further embodiments, the system 1916 may detect the type of subject support device 1912 it is communicating with in the manner described above and then may send a message requiring the user to verify that the system 1916 properly detected the type of subject support device being used by the user. For systems 1916 that are capable of communicating with multiple different types of subject support devices 1912, the device communication layer 1924 may be capable of implementing multiple different communication protocols and selects a protocol that is appropriate for the detected type of subject support device.

The data-parsing layer 1926 is responsible for validating the integrity of device data received and for inputting it correctly into a database 1929. A cyclic redundancy check CRC process for checking the integrity of the received data may be employed. Alternatively, or in addition, data may be received in packets or other data arrangements, where preambles or other portions of the data include device type identification information. Such preambles or other portions of the received data may further include device serial numbers or other identification information that may be used for validating the authenticity of the received information. In such embodiments, the system 1916 may compare received identification information with pre-stored information to evaluate whether the received information is from a valid source.

The database layer 1928 may include a centralized database repository that is responsible for warehousing and archiving stored data in an organized format for later access, and retrieval. The database layer 1928 operates with one or more data storage device(s) 1929 suitable for storing and providing access to data in the manner described herein. Such data storage device(s) 1929 may comprise, for example, one or more hard discs, optical discs, tapes, digital libraries or other suitable digital or analog storage media and associated drive devices, drive arrays or the like.

Data may be stored and archived for various purposes, depending upon the embodiment and environment of use. Information regarding specific subjects and patient support devices may be stored and archived and made available to those specific subjects, their authorized healthcare providers and/or authorized healthcare payor entities for analyzing the subject's condition. Also, certain information regarding groups of subjects or groups of subject support devices may be made available more generally for healthcare providers, subjects, personnel of the entity administering the system 1916 or other entities, for analyzing group data or other forms of conglomerate data.

Embodiments of the database layer 1928 and other components of the system 1916 may employ suitable data security measures for securing personal medical information of subjects, while also allowing non-personal medical information to be more generally available for analysis. Embodiments may be configured for compliance with suitable government regulations, industry standards, policies or the like, including, but not limited to the Health Insurance Portability and Accountability Act of 1996 (HIPAA).

The database layer 1928 may be configured to limit access of each user to types of information pre-authorized for that user. For example, a subject may be allowed access to his or her individual medical information (with individual identifiers) stored by the database layer 1928, but not allowed access to other subject's individual medical information (with individual identifiers). Similarly, a subject's authorized healthcare provider or payor entity may be provided access to some or all of the subject's individual medical information (with individual identifiers) stored by the database layer 1928, but not allowed access to another individual's personal information. Also, an operator or administrator-user (on a separate computer communicating with the computing device 1900) may be provided access to some or all subject information, depending upon the role of the operator or administrator. On the other hand, a subject, healthcare provider, operator, administrator or other entity, may be authorized to access general information of unidentified individuals, groups or conglomerates (without individual identifiers) stored by the database layer 1928 in the data storage devices 1929.

In exemplary embodiments, the database 1929 stores uploaded measurement data for a patient (e.g., sensor glucose measurement and characteristic impedance values) along with event log data consisting of event records created during a monitoring period corresponding to the measurement data. In embodiments of the invention, the database layer 1928 may also store preference profiles. In the database layer 1928, for example, each user may store information regarding specific parameters that correspond to the user. Illustratively, these parameters could include target blood glucose or sensor glucose levels, what type of equipment the users utilize (insulin pump, glucose sensor, blood glucose meter, etc.) and could be stored in a record, a file, or a memory location in the data storage device(s) 1929 in the database layer. Preference profiles may include various threshold values, monitoring period values, prioritization criteria, filtering criteria, and/or other user-specific values for parameters to generate a snapshot GUI display on the display 1933 or a support device 1912 in a personalized or patient-specific manner.

The DDMS 1916 may measure, analyze, and track either blood glucose (BG) or sensor glucose (SG) measurements (or readings) for a user. In embodiments of the invention, the medical data management system may measure, track, or analyze both BG and SG readings for the user. Accordingly, although certain reports may mention or illustrate BG or SG only, the reports may monitor and display results for the other one of the glucose readings or for both of the glucose readings.

The reporting layer 1930 may include a report wizard program that pulls data from selected locations in the database 1929 and generates report information from the desired parameters of interest. The reporting layer 1930 may be configured to generate multiple different types of reports, each having different information and/or showing information in different formats (arrangements or styles), where the type of report may be selectable by the user. A plurality of pre-set types of report (with pre-defined types of content and format) may be available and selectable by a user. At least some of the pre-set types of reports may be common, industry standard report types with which many healthcare providers should be familiar. In exemplary embodiments described herein, the reporting layer 1930 also facilitates generation of a snapshot report including a snapshot GUI display.

In embodiments of the invention, the database layer 1928 may calculate values for various medical information that is to be displayed on the reports generated by the report or reporting layer 1930. For example, the database layer 1928, may calculate average blood glucose or sensor glucose readings for specified timeframes. In embodiments of the invention, the reporting layer 1930 may calculate values for medical or physical information that is to be displayed on the reports. For example, a user may select parameters which are then utilized by the reporting layer 1930 to generate medical information values corresponding to the selected parameters. In other embodiments of the invention, the user may select a parameter profile that previously existed in the database layer 1928.

Alternatively, or in addition, the report wizard may allow a user to design a custom type of report. For example, the report wizard may allow a user to define and input parameters (such as parameters specifying the type of content data, the time period of such data, the format of the report, or the like) and may select data from the database and arrange the data in a printable or displayable arrangement, based on the user-defined parameters. In further embodiments, the report wizard may interface with or provide data for use by other programs that may be available to users, such as common report generating, formatting or statistical analysis programs. In this manner, users may import data from the system 1916 into further reporting tools familiar to the user. The reporting layer 1930 may generate reports in displayable form to allow a user to view reports on a standard display device, printable form to allow a user to print reports on standard printers, or other suitable forms for access by a user. Embodiments may operate with conventional file format schemes for simplifying storing, printing and transmitting functions, including, but not limited to PDF, JPEG, or the like. Illustratively, a user may select a type of report and parameters for the report and the reporting layer 1930 may create the report in a PDF format. A PDF plug-in may be initiated to help create the report and also to allow the user to view the report. Under these operating conditions, the user may print the report utilizing the PDF plug-in. In certain embodiments in which security measures are implemented, for example, to meet government regulations, industry standards or policies that restrict communication of subject's personal information, some or all reports may be generated in a form (or with suitable software controls) to inhibit printing, or electronic transfer (such as a non-printable and/or non-capable format). In yet further embodiments, the system 1916 may allow a user generating a report to designate the report as non-printable and/or non-transferable, whereby the system 1916 will provide the report in a form that inhibits printing and/or electronic transfer.

The reporting layer 1930 may transfer selected reports to the graph display layer 1931. The graph display layer 1931 receives information regarding the selected reports and converts the data into a format that can be displayed or shown on a display 1933.

In embodiments of the invention, the reporting layer 1930 may store a number of the user's parameters. Illustratively, the reporting layer 1930 may store the type of carbohydrate units, a blood glucose movement or sensor glucose reading, a carbohydrate conversion factor, and timeframes for specific types of reports. These examples are meant to be illustrative and not limiting.

Data analysis and presentations of the reported information may be employed to develop and support diagnostic and therapeutic parameters. Where information on the report relates to an individual subject, the diagnostic and therapeutic parameters may be used to assess the health status and relative well-being of that subject, assess the subject's compliance to a therapy, as well as to develop or modify treatment for the subject and assess the subject's behaviors that affect his/her therapy. Where information on the report relates to groups of subjects or conglomerates of data, the diagnostic and therapeutic parameters may be used to assess the health status and relative well-being of groups of subjects with similar medical conditions, such as, but not limited to, diabetic subjects, cardiac subjects, diabetic subjects having a particular type of diabetes or cardiac condition, subjects of a particular age, sex or other demographic group, subjects with conditions that influence therapeutic decisions such as but not limited to pregnancy, obesity, hypoglycemic unawareness, learning disorders, limited ability to care for self, various levels of insulin resistance, combinations thereof, or the like.

The user interface layer 1932 supports interactions with the end user, for example, for user login and data access, software navigation, data input, user selection of desired report types and the display of selected information. Users may also input parameters to be utilized in the selected reports via the user interface layer 1932. Examples of users include but are not limited to: healthcare providers, healthcare payer entities, system operators or administrators, researchers, business entities, healthcare institutions and organizations, or the like, depending upon the service being provided by the system and depending upon the invention embodiment. More comprehensive embodiments are capable of interacting with some or all of the above-noted types of users, wherein different types of users have access to different services or data or different levels of services or data.

In an example embodiment, the user interface layer 1932 provides one or more websites accessible by users on the Internet. The user interface layer may include or operate with at least one (or multiple) suitable network server(s) to provide the website(s) over the Internet and to allow access, world-wide, from Internet-connected computers using standard Internet browser software. The website(s) may be accessed by various types of users, including but not limited to subjects, healthcare providers, researchers, business entities, healthcare institutions and organizations, payor entities, pharmaceutical partners or other sources of pharmaceuticals or medical equipment, and/or support personnel or other personnel running the system 1916, depending upon the embodiment of use.

In another example embodiment, where the DDMS 1916 is located on one computing device 1900, the user interface layer 1932 provides a number of menus to the user to navigate through the DDMS. These menus may be created utilizing any menu format, including but not limited to HTML, XML, or Active Server pages. A user may access the DDMS 1916 to perform one or more of a variety of tasks, such as accessing general information made available on a website to all subjects or groups of subjects. The user interface layer 1932 of the DDMS 1916 may allow a user to access specific information or to generate reports regarding that subject's medical condition or that subject's medical device(s) 1912, to transfer data or other information from that subject's support device(s) 1912 to the system 1916, to transfer data, programs, program updates or other information from the system 1916 to the subject's support device(s) 1912, to manually enter information into the system 1916, to engage in a remote consultation exchange with a healthcare provider, or to modify the custom settings in a subject's supported device and/or in a subject's DDMS/MDMS data file.

The system 1916 may provide access to different optional resources or activities (including accessing different information items and services) to different users and to different types or groups of users, such that each user may have a customized experience and/or each type or group of user (e.g., all users, diabetic users, cardio users, healthcare provider-user or payor-user, or the like) may have a different set of information items or services available on the system. The system 1916 may include or employ one or more suitable resource provisioning program or system for allocating appropriate resources to each user or type of user, based on a pre-defined authorization plan. Resource provisioning systems are well known in connection with provisioning of electronic office resources (email, software programs under license, sensitive data, etc.) in an office environment, for example, in a local area network LAN for an office, company or firm. In one example embodiment, such resource provisioning systems is adapted to control access to medical information and services on the DDMS 1916, based on the type of user and/or the identity of the user.

Upon entering successful verification of the user's identification information and password, the user may be provided access to secure, personalized information stored on the DDMS 1916. For example, the user may be provided access to a secure, personalized location in the DDMS 1916 which has been assigned to the subject. This personalized location may be referred to as a personalized screen, a home screen, a home menu, a personalized page, etc. The personalized location may provide a personalized home screen to the subject, including selectable icons or menu items for selecting optional activities, including, for example, an option to transfer device data from a subject's supported device 1912 to the system 1916, manually enter additional data into the system 1916, modify the subject's custom settings, and/or view and print reports. Reports may include data specific to the subject's condition, including but not limited to, data obtained from the subject's subject support device(s) 1912, data manually entered, data from medical libraries or other networked therapy management systems, data from the subjects or groups of subjects, or the like. Where the reports include subject-specific information and subject identification information, the reports may be generated from some or all subject data stored in a secure storage area (e.g., storage devices 1929) employed by the database layer 1928.

The user may select an option to transfer (send) device data to the medical data management system 1916. If the system 1916 receives a user's request to transfer device data to the system, the system 1916 may provide the user with step-by-step instructions on how to transfer data from the subject's supported device(s) 1912. For example, the DDMS 1916 may have a plurality of different stored instruction sets for instructing users how to download data from different types of subject support devices, where each instruction set relates to a particular type of subject supported device (e.g., pump, sensor, meter, or the like), a particular manufacturer's version of a type of subject support device, or the like. Registration information received from the user during registration may include information regarding the type of subject support device(s) 1912 used by the subject. The system 1916 employs that information to select the stored instruction set(s) associated with the particular subject's support device(s) 1912 for display to the user.

Other activities or resources available to the user on the system 1916 may include an option for manually entering information to the DDMS/MDMS 1916. For example, from the user's personalized menu or location, the user may select an option to manually enter additional information into the system 1916.

Further optional activities or resources may be available to the user on the DDMS 1916. For example, from the user's personalized menu, the user may select an option to receive data, software, software updates, treatment recommendations or other information from the system 1916 on the subject's support device(s) 1912. If the system 1916 receives a request from a user to receive data, software, software updates, treatment recommendations or other information, the system 1916 may provide the user with a list or other arrangement of multiple selectable icons or other indicia representing available data, software, software updates or other information available to the user.

Yet further optional activities or resources may be available to the user on the medical data management system 1916 including, for example, an option for the user to customize or otherwise further personalize the user's personalized location or menu. In particular, from the user's personalized location, the user may select an option to customize parameters for the user. In addition, the user may create profiles of customizable parameters. When the system 1916 receives such a request from a user, the system 1916 may provide the user with a list or other arrangement of multiple selectable icons or other indicia representing parameters that may be modified to accommodate the user's preferences. When a user selects one or more of the icons or other indicia, the system 1916 may receive the user's request and makes the requested modification.

Infusion System Overview

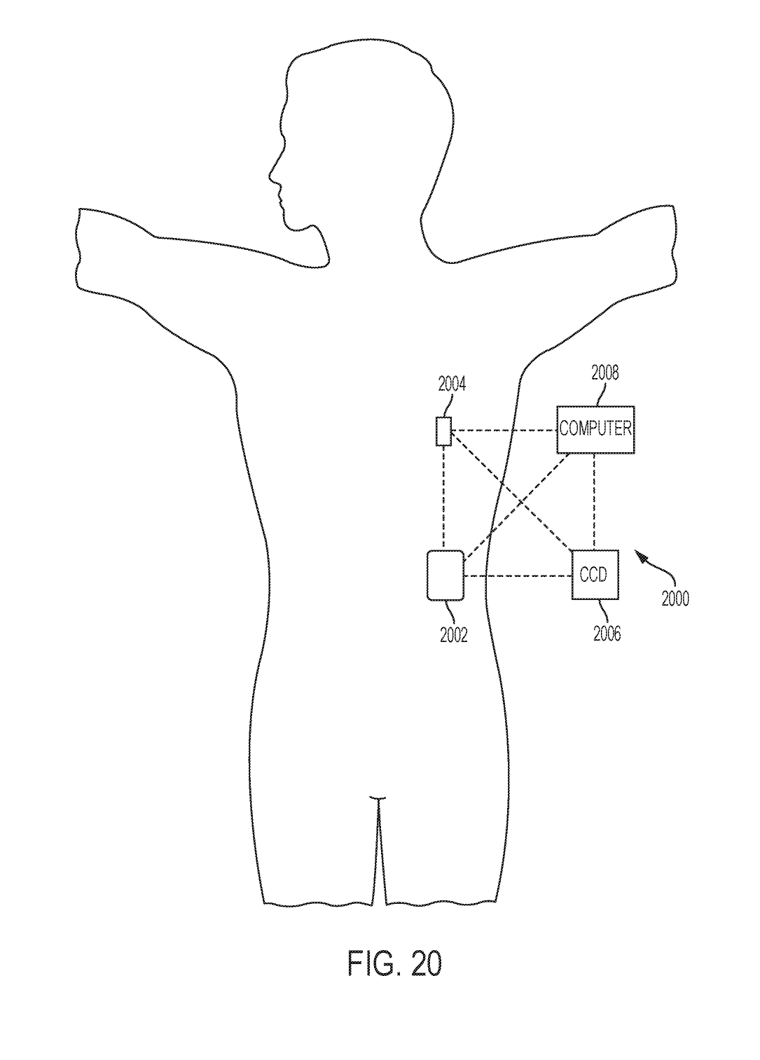

Referring now to FIG. 20, in some embodiments, the data transfer process 200 and/or the event monitoring process 600 may be implemented in connection with an infusion system 2000 that includes, without limitation, a fluid infusion device (or infusion pump) 2002, a sensing arrangement 2004 (e.g. monitoring device 102), a command control device (CCD) 2006 (e.g., client device 106), and a computer 2008 (e.g., server 114). The components of an infusion system 2000 may be realized using different platforms, designs, and configurations, and the embodiment shown in FIG. 20 is not exhaustive or limiting. In practice, the infusion device 2002 and the sensing arrangement 2004 are secured at desired locations on the body of a user (or patient), as illustrated in FIG. 20. In this regard, the locations at which the infusion device 2002 and the sensing arrangement 2004 are secured to the body of the user in FIG. 20 are provided only as a representative, non-limiting, example. The elements of the infusion system 2000 may be similar to those described in U.S. Pat. No. 8,674,288, the subject matter of which is hereby incorporated by reference in its entirety.

In the illustrated embodiment of FIG. 20, the infusion device 2002 is designed as a portable medical device suitable for infusing a fluid, a liquid, a gel, or other agent into the body of a user. In exemplary embodiments, the infused fluid is insulin, although many other fluids may be administered through infusion such as, but not limited to, HIV drugs, drugs to treat pulmonary hypertension, iron chelation drugs, pain medications, anti-cancer treatments, medications, vitamins, hormones, or the like. In some embodiments, the fluid may include a nutritional supplement, a dye, a tracing medium, a saline medium, a hydration medium, or the like.

The sensing arrangement 2004 generally represents the components of the infusion system 2000 configured to sense, detect, measure or otherwise quantify a condition of the user, and may include a sensor, a monitor, or the like, for providing data indicative of the condition that is sensed, detected, measured or otherwise monitored by the sensing arrangement. In this regard, the sensing arrangement 2004 may include electronics and enzymes reactive to a biological or physiological condition of the user, such as a blood glucose level, or the like, and provide data indicative of the blood glucose level to the infusion device 2002, the CCD 2006 and/or the computer 2008. For example, the infusion device 2002, the CCD 2006 and/or the computer 2008 may include a display for presenting information or data to the user based on the sensor data received from the sensing arrangement 2004, such as, for example, a current glucose level of the user, a graph or chart of the user's glucose level versus time, device status indicators, alert messages, or the like. In other embodiments, the infusion device 2002, the CCD 2006 and/or the computer 2008 may include electronics and software that are configured to analyze sensor data and operate the infusion device 2002 to deliver fluid to the body of the user based on the sensor data and/or preprogrammed delivery routines. Thus, in exemplary embodiments, one or more of the infusion device 2002, the sensing arrangement 2004, the CCD 2006, and/or the computer 2008 includes a transmitter, a receiver, and/or other transceiver electronics that allow for communication with other components of the infusion system 2000, so that the sensing arrangement 2004 may transmit sensor data or monitor data to one or more of the infusion device 2002, the CCD 2006 and/or the computer 2008.

Still referring to FIG. 20, in various embodiments, the sensing arrangement 2004 may be secured to the body of the user or embedded in the body of the user at a location that is remote from the location at which the infusion device 2002 is secured to the body of the user. In various other embodiments, the sensing arrangement 2004 may be incorporated within the infusion device 2002. In other embodiments, the sensing arrangement 2004 may be separate and apart from the infusion device 2002, and may be, for example, part of the CCD 2006. In such embodiments, the sensing arrangement 2004 may be configured to receive a biological sample, analyte, or the like, to measure a condition of the user.

In various embodiments, the CCD 2006 and/or the computer 2008 may include electronics and other components configured to perform processing, delivery routine storage, and to control the infusion device 2002 in a manner that is influenced by sensor data measured by and/or received from the sensing arrangement 2004. By including control functions in the CCD 2006 and/or the computer 2008, the infusion device 2002 may be made with more simplified electronics. However, in other embodiments, the infusion device 2002 may include all control functions, and may operate without the CCD 2006 and/or the computer 2008. In various embodiments, the CCD 2006 may be a portable electronic device. In addition, in various embodiments, the infusion device 2002 and/or the sensing arrangement 2004 may be configured to transmit data to the CCD 2006 and/or the computer 2008 for display or processing of the data by the CCD 2006 and/or the computer 2008.

In some embodiments, the CCD 2006 and/or the computer 2008 may provide information to the user that facilitates the user's subsequent use of the infusion device 2002. For example, the CCD 2006 may provide information to the user to allow the user to determine the rate or dose of medication to be administered into the user's body. In other embodiments, the CCD 2006 may provide information to the infusion device 2002 to autonomously control the rate or dose of medication administered into the body of the user. In some embodiments, the sensing arrangement 2004 may be integrated into the CCD 2006. Such embodiments may allow the user to monitor a condition by providing, for example, a sample of his or her blood to the sensing arrangement 2004 to assess his or her condition. In some embodiments, the sensing arrangement 2004 and the CCD 2006 may be used for determining glucose levels in the blood and/or body fluids of the user without the use of, or necessity of, a wire or cable connection between the infusion device 2002 and the sensing arrangement 2004 and/or the CCD 2006.

In one or more exemplary embodiments, the sensing arrangement 2004 and/or the infusion device 2002 are cooperatively configured to utilize a closed-loop system for delivering fluid to the user. Examples of sensing devices and/or infusion pumps utilizing closed-loop systems may be found at, but are not limited to, the following U.S. Pat. Nos. 6,088,608, 6,119,028, 6,589,229, 6,740,072, 6,827,702, 7,323,142, and 7,402,153, all of which are incorporated herein by reference in their entirety. In such embodiments, the sensing arrangement 2004 is configured to sense or measure a condition of the user, such as, blood glucose level or the like. The infusion device 2002 is configured to deliver fluid in response to the condition sensed by the sensing arrangement 2004. In turn, the sensing arrangement 2004 continues to sense or otherwise quantify a current condition of the user, thereby allowing the infusion device 2002 to deliver fluid continuously in response to the condition currently (or most recently) sensed by the sensing arrangement 2004 indefinitely. In some embodiments, the sensing arrangement 2004 and/or the infusion device 2002 may be configured to utilize the closed-loop system only for a portion of the day, for example only when the user is asleep or awake.

The monitoring client application 108 and the processes 200, 600 described herein facilitate a diabetic patient electronically logging their behavior during a monitoring period for integration with concurrent measurement data to facilitate retrospective analysis of the patient's lifestyle activities in concert with the patient's sensed glucose levels during the monitoring period. For example, the patient's doctor or other healthcare provider may review a snapshot GUI display or similar display that includes a graph of the patient's sensed glucose values over a monitoring period with overlaid indicators corresponding to manually-entered events from the journal or event log maintained by the patient via the monitoring client application 108 during the study. In some embodiments, the monitoring client application 108 and the processes 200, 600 may be utilized for purposes of a patient study to assess the patient's suitability for an infusion pump and closed-loop glucose control. When used in connection with an insulin infusion pump, the monitoring client application 108 and the processes 200, 600 may be utilized to analyze the efficacy of the glucose regulation achieved by the closed-loop control scheme and adjust controller targets, basal infusion rates, or other patient-specific parameters.

Event Log GUI Display Examples

FIG. 21 depicts an exemplary embodiment of a graph overlay region 2100 that may be presented on or by an electronic device in connection with the subject matter described herein. In one or more embodiments, the graph overlay region 2100 is presented at the bottom of a snapshot GUI display, such as one of those provided in U.S. Patent Pub. No. 2017/0106144. The graph overlay region 2100 includes a graphical representation 2102 of historical measurement data for the patient's glucose level over the snapshot time period with respect to time along with a visually distinguishable overlay region that indicates a target range for the patient's sensor glucose measurement values. The illustrated graph overlay region 2100 also includes a legend 2104 indicating and explaining the different graphical indicia for the different event types that could be recorded as part of a patient's event log data. The graphical indicia for a particular event type may also utilize one or more visually distinguishable characteristics to indicate the relative intensity, size, or other characteristic associated with a particular event. For example, different amounts of shading or fill may be applied to an exercise event icon to indicate the relative amount of intensity of an exercise event. In this regard, the descriptive information associated with an event may be utilized to select the appropriate graphical indicator and visually distinguishable characteristics for depicting the event.

Using the stored event log data associated with a patient, a plurality of graphical indicia 2110, 2112, 2114, 2116, 2118, 2120, 2122 corresponding to the events logged by that patient may be presented on the graph overlay region 2100 in association with the graphical representation of the patient's sensor glucose measurement data 2102. For example, an unfilled meal icon 2110 indicative of consumption of a small meal by the patient may be presented on the graph overlay region 2100 at a temporal location corresponding to the time associated with the small meal that was input by the patient (e.g., via regions 1102 and 1104). Similarly, an insulin injection icon 2112 and a medication icon 2114 are presented on the graph overlay region 2100 at a temporal location corresponding to the times that were input by the patient (e.g., via region 1302). A partially-filled meal icon 2116 indicative of consumption of a moderately-sized meal by the patient is presented on the graph overlay region 2100 at a temporal location corresponding to the input time associated with the moderately-sized meal, and a filled meal icon 2120 indicative of consumption of a large meal by the patient is presented on the graph overlay region 2100 at a temporal location corresponding to the input time associated with the large meal. A sleep icon 2122 may also be presented at a time associated with a bedtime input by the patient.

A partially-filled exercise icon 2118 indicative of moderate intensity exercise is presented on the graph overlay region 2100 at a temporal location corresponding to the input time associated with the exercise. Additionally, the descriptive information associated with the moderate intensity exercise event may be analyzed to identify or otherwise determine the duration associated with the exercise event and provide graphical indicia 2119 of the exercise duration in concert with the exercise icon 2118. For example, the exercise icon 2118 may be located at an end time associated with the exercise event with the exercise duration indicia 2119 being realized as a graphical representation of a trail following the exercise icon 2118 that extends from the end time to the start time associated with the exercise event. Thus, the indicia 2118, 2119 operate in concert to provide an indication of both the duration and intensity of an input exercise event on the graph overlay region 2100.

FIG. 22 depicts an exemplary embodiment of a graph overlay region 2200 that includes graphical annotations presented in concert with the event type indicia. In this regard, input descriptive information associated with an event may be analyzed to identify or otherwise determine parameters or values for characteristics that further quantify or qualify the event. For example, a carbohydrate amount annotation 2210 may be presented in concert with a meal event icon 2110 to indicate the amount of carbohydrates associated with that meal, which were input by the patient or estimated based on the meal size and type of food consumed. A dosage annotation 2212 indicating the input bolus dosage amount (e.g., via region 1304) is presented in concert with the insulin injection icon 2112. Based on the duration and intensity associated with an exercise event and other patient physiological information (e.g., stored in database 116), an estimated amount of calories burned during the exercise event may be calculated (e.g., by the server 114) and presented as an annotation 2218 associated with an exercise icon 2118 for the exercise event. As another example, an annotation 2220 indicating a type of food consumed may be presented in concert with a meal indicator 2120. A sleep duration for the patient may be calculated based on a difference between a start time associated with the patient's bed time and a wake-up time (which could be manually indicated or calculated based on sensor data or other factors) and presented as a sleep duration annotation 2222 proximate a sleep icon 2122. In some embodiments, a duration indicia similar to the exercise duration indicia 2119 may be presented on a graph overlay region to indicate the sleep duration.

It should be noted that FIGS. 21-22 merely depict some exemplary graphical indicia that may be presented based on event log data and descriptive information associated with logged events, and the subject matter described herein is not limited to any particular graphical indicia or GUI displays for presenting event log data. In this regard, the graphical event indicia are not limited to presentation on a snapshot GUI display.

Automated Display Toggling

FIG. 23 depicts an exemplary display toggling process 2300 suitable for implementation in patient monitoring system to periodically present measurement data for a patient's physiological condition being monitored in an automated or autonomous manner. In this regard, the display toggling process 2300 may be performed concurrently to or in concert with the data transfer process 200 of FIG. 2 and/or the event monitoring process 600 of FIG. 6, for example, to automatically and autonomously toggle to or from a home screen GUI display (e.g., home screen GUI display 1800) where measurement data is invisible. The various tasks performed in connection with the display toggling process 2300 may be performed by hardware, firmware, software executed by processing circuitry, or any combination thereof. For purposes of explanation, the display toggling process 2300 may be described herein as primarily being performed or supported by the client application 108 at the client device 106. It should be appreciated that the display toggling process 2300 may include any number of additional or alternative tasks, the tasks need not be performed in the illustrated order and/or the tasks may be performed concurrently, and/or the display toggling process 2300 may be incorporated into a more comprehensive procedure or process having additional functionality not described in detail herein. Moreover, one or more of the tasks shown and described in the context of FIG. 23 could be omitted from a practical embodiment of the display toggling process 2300 as long as the intended overall functionality remains intact.

In exemplary embodiments, the display toggling process 2300 is performed to automatically toggle a GUI display provided by the client application 108 at the client device 106 between a normal display state (or mode) where measurement data is hidden or otherwise invisible and a diagnostic display state (or mode) where measurement data is presented on a display associated with the client device 106. It should be noted that while the subject matter is primarily described herein in the context of the display toggling process 2300 being performed with respect to a display on the client device 106, the display toggling process 2300 is not necessarily limited to the client device 106 and may be implemented in an equivalent manner to toggle a display associated with the monitoring device 102 or another device 114, 1900, 1912, 2008, 2010 capable of receiving measurement data from the monitoring device 102 and/or sensing element 104.

The display toggling process 2300 initializes by hiding or otherwise preventing measurement data obtained from the monitoring device from being displayed to a user (task 2302). For example, as described above, the client device 106 may autonomously obtain measurement data from the monitoring device 102 and upload the measurement data to the remote device 114 in a manner that is invisible to the user and without displaying or presenting the measurement data at the client device 106. In the normal display mode, a home screen GUI display may be presented by the client application 108 on the client device 106, with the measurement data being hidden or prevented from presentation on the home screen GUI display.

While the measurement data is maintained invisible to the patient in the normal display mode, the display toggling process 2300 monitors for a condition configured to trigger toggling the display to present measurement data (task 2304). In response to detecting or otherwise identifying a condition triggering toggling of the display, the display toggling process 2300 automatically updates, switches, or toggles the display from the normal display mode to a diagnostic display mode to present the current measurement data provided by the monitoring device (task 2306). As described in greater detail below, the client application 108 may be configurable to identify or detect any number of different potential conditions that may be utilized to trigger a change in the display state of the client application 108. In some embodiments, the client application 108 automatically switches to the diagnostic display mode to present measurement data from the monitoring device 102 substantially immediately in response to detecting a display toggling condition. However, in other embodiments, the client application 108 may be configured to wait for a delay period (e.g., fifteen minutes) before changing the display at the client device 106. In this regard, in some embodiments, the client application 108 may be configured to verify that the display toggling condition (e.g., a high or low glucose excursion) persists or otherwise still exists after the delay period before changing the display state to mitigate transients, noise or other artifacts.

In one or more embodiments, in response to detecting a toggling condition for transitioning into the diagnostic display mode, the client application 108 transmits or otherwise provides a request for the current or most recent measurement data from the monitoring device 102. For example, as described above, in one or more exemplary embodiments, measurement data is not stored at the client device 106 in the normal display mode. Rather, the monitoring device 102 buffers, stores, or otherwise maintains measurement data samples for a preceding period of time until determining a threshold amount of measurement data is available for uploading (e.g., at least 6 hours' worth) before notifying the client application 108 of available measurement data. Accordingly, the client application 108 may request at least the current or most recent measurement data sample from the monitoring device 102 in order to generate a graphical representation of measurement data at the client device 106. Depending on the type of diagnostic display to be generated at the client device 106, the client application 108 may request additional measurement data samples for a preceding window of time to be depicted on the client device 106. For example, if the diagnostic display includes a graphical representation of patient's measured sensor glucose levels over a preceding time interval for retrospective analysis, the client application 108 may request the most recent measurement data sample available at the monitoring device 102 and preceding measurement data samples timestamped or obtained within the preceding time interval (e.g., any measurement data samples having timestamps within the last two hours).

Additionally or alternatively, in some embodiments, in response to detecting a toggling condition, the client application 108 may request measurement data or other data (e.g., event log data) from the remote device 114 which may be utilized to generate or augment a diagnostic display at the client device 106. For example, the client application 108 may request event log data from the remote device 114 for generating event type indicia or other event log information on or overlying a representation of measurement data obtained from the monitoring device 102. Additionally, in embodiments where sufficient historical measurement data samples are not available from the monitoring device 102 for generating the retrospective measurement data diagnostic display (e.g., in embodiments where measurement data samples are deleted, discarded, or overwritten at the monitoring device 102 after being uploaded to the remote device 114), the client application 108 requests additional measurement data samples for a preceding window of time to be depicted on the client device 106 from the remote device 114. Thus, the graphical representation of measurement data may include depictions of recent measurement data obtained from the monitoring device 102 augmented with older measurement data obtained from the database 116 via the remote device 114.

In one or more embodiments, a diagnostic display provided by the client application 108 at the client device 106 may include graphical representations of predicted or anticipated glucose levels for the patient in addition to the current or most recent sensed glucose level. In such embodiments, previous measurement data samples may be analyzed by any one of the devices 102, 106, 114 to identify one or more trends in the patient's glucose level, which, in turn, may be utilized to calculate or otherwise estimate predicted glucose levels at different times in the future. Thus, depending on the embodiment, a diagnostic display at the client device 106 may depict the patient's current or most recent sensor glucose value concurrently to presenting one or more preceding sensor glucose values in the past and/or one or more predicted sensor glucose values at different points in the future.

Still referring to FIG. 23, in exemplary embodiments, the display toggling process 2300 maintains presentation of measurement data until identifying or otherwise determining that the diagnostic display mode should be terminated, at which point the display toggling process 2300 reverts to hiding measurement data from the patient (tasks 2308, 2310). In one or more embodiments, the client application 108 implements a timer or similar feature to limit the duration of time that the measurement data is presented to the patient to a fixed time period. In this regard, in various embodiments, the fixed time period may vary depending on the type of toggling condition detected (e.g., at task 2304). For example, for some toggling conditions, it may be desirable to allow the patient to monitor his or her glucose levels for a longer duration of time relative to other toggling conditions having different glycemic effects. In other embodiments, the client application 108 may maintain the diagnostic display state until detecting another condition configured to trigger toggling the display from presenting measurement data to hiding measurement data. In this regard, the toggling condition that triggers transitioning from a diagnostic display mode back to a normal display mode may be different from the toggling condition that previously triggered the transition from the normal display mode to the diagnostic display mode. In some embodiments, the absence of the toggling condition that previously triggered the transition from the normal display mode to the diagnostic display mode may be detected and utilized to trigger reverting the display from the diagnostic display mode back to the normal display mode.

In the absence of the diagnostic display mode timing out or another toggling condition being detected, the loop defined by tasks 2306 and 2308 may be repeated indefinitely to dynamically update the measurement data presented on the diagnostic display. In this regard, in some embodiments, the client application 108 may periodically poll the monitoring device 102 for a newer or updated measurement data sample, which, in turn may be utilized to update the display at the client device 106 substantially in real-time. In yet other embodiments, the client application 108 may command, signal, or otherwise instruct the monitoring device 102 to enter a real-time mode where the monitoring device 102 is configured to automatically push new measurement data samples to the client device 106 when they are available. For example, each time a new measurement data sample becomes available in the real-time mode, the monitoring device 102 may automatically set the flag that causes the client device 106 to retrieve the measurement data sample from the monitoring device 102 in a similar manner as described above in the context of FIG. 2.

After reverting to the normal display mode where measurement data is hidden, the display toggling process 2300 may repeat for the duration of a study or monitoring period. In this regard, the display toggling process 2300 may cause the client application 108 to autonomously toggle the display at the client device 106 at multiple different times during the study, for potentially different reasons and for potentially different durations. Thus, the selective visibility of the measurement data may be utilized to encourage, discourage, or otherwise influence patient behavior as desired for purposes of a study by adjusting or tailoring the display toggling criteria for the particular study or patient.

For example, in one or more embodiments, the client application 108 is configured to monitor the patient's behavior during a study to identify or detect toggling conditions based on events logged by the patient or other aspects of the patient's behavior. For example, a meal event could be utilized as one criterion defining a toggling condition for transitioning from a normal display mode to a diagnostic display mode. In response to receiving input from the patient logging contemporaneous consumption of a meal, administration of a meal bolus, or the like, the client application 108 identifies or otherwise detects a meal event and automatically obtains at least the most recent sensor glucose value from the monitoring device 102 updates the GUI display at the client device 106 to include a graphical representation of the patient's most recent sensor glucose value(s). As another example, an exercise event could be utilized as a toggling condition for transitioning from a normal display mode to a diagnostic display mode. In response to receiving input from the patient logging an exercise event or otherwise detecting exercise (e.g., based on the output of an acceleration sensing arrangement, a heart rate monitor, or the like), the client application 108 may automatically transition from the normal display mode to a diagnostic display mode.

In some embodiments, the display toggling process 2300 may be utilized to encourage or discourage a particular type of patient behavior. For example, in some embodiments, a patient may be rewarded for obtaining blood glucose measurements by transitioning from a normal display mode to a diagnostic display mode in response to a new blood glucose reading being logged, received, or otherwise detected. In this regard, the threshold duration of time for maintaining the diagnostic display mode may be configured to encourage obtaining new blood glucose measurements with a desired frequency, so that the client application 108 automatically reverts from the diagnostic display mode to the normal display mode once a new blood glucose measurement is required to maintain the desired frequency of blood glucose measurements. For example, in response to an absence of a new blood glucose measurement from a blood glucose meter within the threshold period of time after a preceding blood glucose measurement, the client application 108 may automatically revert to hiding real-time sensor glucose measurement data until a new blood glucose measurement value is obtained. In a similar manner, the display toggling process 2300 may be utilized to encourage or discourage exercise or other behaviors with a desired frequency.

In one or more embodiments, patient behavior considered to be good or desirable can be rewarded by toggling the display to provide an image or other content deemed favorable to the user, such as, for example, by presenting one or more of the patient's favorite pictures available in his or her photo library. As another example, textual or graphical content may be provided that encourages the patient to advance his or her monitoring. In this regard, different qualitative levels of patient status corresponding to different behavioral characteristics may be supported, with content being provided on the display that encourages the patient to engage in good behavior that will advance the patient's current level. For example, different patient levels or categories (e.g., beginner, intermediate, advanced, and/or the like) may correspond to different durations of time or percentages of the day during which the real-time sensor glucose measurement data is presented to the particular patient, with the displayed content providing feedback to the patient that indicates the patient's current level or status and suggests or otherwise encourages the patient to engage in desirable behaviors that are likely to advance the patient from the current level. Thus, a beginner level patient may be provided a certain type of feedback that indicates how the patient can advance to a higher level, while higher level patients may be provided with different types of feedback according to his or her behavioral characteristics and current patient level. Moreover, feedback indicating advancement or change in the patient's level or status may be provided in concert with the real-time measurement data display as a reward for the patient in response to detecting a change in the patient's level or status.

In one or more exemplary embodiments, the display toggling process 2300 is configured to monitor one or more aspects of the patient's behavior to determine whether the patient's behavior during the diagnostic display mode deviates from the patient's nominal behavior during the normal display mode in a manner that indicates the patient is attempting to manipulate, influence, or game the study, and if so, the client application 108 may automatically revert from the diagnostic display mode to the normal display mode to discourage such behavioral changes. For example, the client application 108 may analyze the patient's event log data and identify changes in exercise habits (e.g., changes in exercise type, duration, intensity, frequency, and/or the like) or meal habits (e.g., changes in meal size, meal type, meal frequency, and/or the like) during periods of time when the client application 108 is in the diagnostic display mode that indicate the patient is undesirably altering his or her behavior to influence the study when the measurement data is presented. In response to identifying a deviation in behavior, the client application 108 may automatically transition back to the normal display state to hide the measurement data from the patient.

In one or more embodiments, the display toggling process 2300 may utilize network connectivity as a display toggling criterion. For example, in one embodiment, in response to an absence of connectivity to the network 112 and/or remote device 114, the client application 108 may automatically transition from the normal display mode to the diagnostic display mode to depict the patient's measurement data. In this regard, since the network 112 and/or remote device 114 is unavailable for monitoring the patient's physiological condition, the client application 108 may automatically present the patient's measurement data so that the patient or other user can manually monitor the patient's physiological condition at the client device 106 in lieu of reliance on remote monitoring. When connectivity to the network 112 and/or remote device 114 is restored, the client application 108 may then automatically revert from the diagnostic display mode to the normal display mode.

In another embodiment, the display toggling process 2300 may utilize the location of the client device 106 as a display toggling criterion. For example, the client application 108 may obtain a geographic location of the client device 106 via a global positioning system (GPS) receiver or similar arrangement and determine whether to transition the display state based on that geographic location. For example, in response to determining the geographic location of the client device 106 is located within a threshold distance of a location of interest or a particular type of location of interest, the client application 108 may automatically toggle the display state to encourage or discourage a particular type of behavior by the patient in connection with that location of interest. For example, if the geographic location of the client device 106 is near a restaurant, a grocery store, or the like, the client application 108 may automatically transition from the normal display mode to the diagnostic display mode to allow the patient to make a decision about whether or not to consume carbohydrates or a meal based on his or her current physiological condition. Similarly, if the geographic location of the client device 106 is near a park or other recreational location, the client application 108 may automatically transition from the normal display mode to the diagnostic display mode to allow the patient to make a decision about whether or not to engage in exercise or other activity.

In one or more embodiments, the display toggling process 2300 may utilize the patient's measurement data as a display toggling criterion. For example, when the patient's sensor glucose level is above a hyperglycemic threshold value or below a hypoglycemic threshold value, the client application 108 may automatically toggle the display state from the normal display mode to the diagnostic display mode to apprise the patient of his or her current physiological condition. Once the patient's sensor glucose level returns to a level that is below the hyperglycemic threshold value and above the hypoglycemic threshold value, the client application 108 may automatically toggle the display state from the diagnostic display mode to the normal display mode to resume hiding the measurement data from the patient. In another embodiment, the client application 108 may automatically toggle the display state from the normal display mode to the diagnostic display mode when the patient's current sensor glucose level is not within a range of sensor glucose values about a target glucose level for the patient, and automatically revert the display state to the normal display mode when the patient's current sensor glucose level returns to being within the desired measurement range about the patient's target glucose level. In some embodiments, the remote device 114 may analyze the patient's historical measurement data to detect or otherwise identify patterns associated with the patient's measurement data (e.g., glucose excursions after a meal), which, in turn may be utilized to configure the display toggling process 2300 to automatically toggle the display state in response to detecting measurement data indicative of a particular pattern of glucose levels that has been defined as a toggling condition.

In one embodiment, the display toggling process 2300 is configured to ensure a desired duration of the study has taken place with the measurement data hidden before enabling toggling to a diagnostic display mode. For example, the display toggling process 2300 may be configured to automatically toggle the display state once measurement data for at least one overnight period has been captured in the normal display mode before transitioning to the diagnostic display mode.

It should be noted that the above are merely some examples of display toggling criteria, and the subject matter described herein is not intended to be limited to any particular type or combination of conditions for automatically toggling the display at the client device 106. For example, various combinations or subsets of measurement data and event log data may be configured to toggle the display state from one state to another. Moreover, various types or combinations of patient data, device status data (e.g., network connectivity, geographic location, remaining battery life, and the like), and potentially other clinical data or information may be utilized to define different conditions for toggling the display state to encourage or discourage various types of behavior, or to otherwise apprise the patient of his or her physiological condition when clinically relevant to the patient's behavior.

Table 1 represents an exemplary set of display toggling criteria suitable for use with the display toggling process 2300 of FIG. 23. It should be noted that Table 1 merely depicts one particular example of display toggling criteria, the subject matter described herein is not limited to the examples provided in Table 1.

TABLE 1

| Real-time/Blinded | | | |
|---|---|---|---|
| Toggle Criteria | Examples | | |
| Patient behavior | 1. | Based on activity/events. Some examples: | |
| during study | | a. | Patient just had a meal, show real-time |
| | | | measurement data (e.g. within 1-2 hours |
| | | | when patient most receptive). Rest of |
| | | | the time, keep blinded. |
| | | b. | Patient just went for a run, show real- |
| | | | time measurement data after (when |
| | | | clinically relevant), but not during run. |
| | | c. | Patient reaches a certain number of steps, |
| | | | toggle real-time/blinded mode |

| | | |
|---|---|---|
| | 2. | Compliance. e.g. If not compliant with BG |
| | | readings, keep retrospective. |
| | 3. | Identify whether patient is “gaming system”. |
| | | Test blinded vs real-time and see whether there |
| | | is behavior change. If not gaming system, use |
| | | real-time measurement data display mode. |
| Connectiv- | a. | If no wifi access/connectivity to remote server, |
| ity/location | | real-time measurement data display mode |
| | b. | If no Bluetooth connectivity for sustained period |
| | | of time, blinded for battery savings |
| | c. | Real-time measurement data display mode |
| | | triggered based on location |

| | |
|---|---|
| i. | Near certain restaurants/grocery stores, |
| | make recommendations on meals/recipes |
| | and turn on real-time to motivate |
| | behavior. |
| ii. | If near park, tell patient to jog (since they |
| | are so close) and turn on real-time |
| | measurement data display. |

| | | |
|---|---|---|
| | d. | Mode dependent on geographies that may |
| | | potentially decide not to reimburse blinded/real- |
| | | time |
| Health care | a. | If using HCP mode, will be blinded. If patient |
| provider | | mode, real-time measurement data display |
| (HCP)/patient | | |
| mode | | |
| Based on sensor | a. | System detects a low or extreme high, go into |
| glucose (SG) | | real-time measurement data display mode |
| data/pattern | b. | System detects patterns (e.g. pattern of |
| detection | | excursions after meals), go to real-time |
| | | measurement data display mode |
| | c. | When inside SG range, blinded. When outside |
| | | SG range, real-time measurement data display |
| After threshold | a. | Based on whether enough weekdays/weeknights |
| number of days, | | are captured during the study in blinded mode. |
| switch from | | If enough (e.g. at least one blinded weeknight), |
| blinded to real- | | go into real-time measurement data display |
| time measurement | | mode |
| data display | b. | During sleep, keep blinded |
| | c. | Decided by the HCP, either prior/during study |
| | d. | Preset by system administrator or provider |
| | e. | Patient decision (e.g. during study via button) |
| Those more open | a. | Based on clinical/behavioral science, turn on |
| to behavior | | real-time measurement data display mode (e.g. |
| modification. | | patients who are younger vs. older, education |
| | | level) |
| | b. | Certifications/training modules completed |
| Care part- | a. | HCP views real-time measurement data display, |
| ners/social/gam- | | while patient is in blinded |
| ification | b. | Friend challenges/asks another person on |
| | | continuous glucose monitoring (CGM) to view |
| | | data. Goes from blinded to real-time |
| | | measurement data display mode. |
| | c. | See someone else's real-time measurement data |
| | | display while they are in blinded mode |
| | d. | If patient meets a challenge (e.g. accumulates |
| | | points for exercise), switch real-time |
| | | measurement data display to blinded |
| | e. | Guessing game: E.g. system asks “what do you |
| | | think your SG will be?”. Switch to real-time |
| | | measurement data display depending on answer |

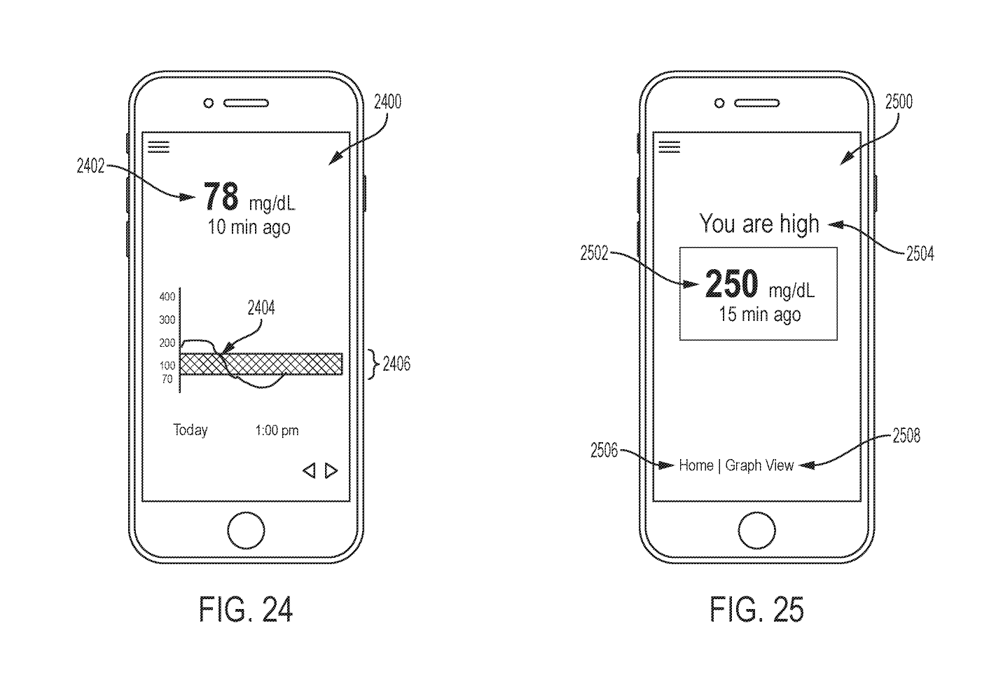

FIG. 24-25 example diagnostic GUI displays 2400, 2500 that may be presented by the client application 108 on the client device 106 in connection with the display toggling process 2300. In this regard, the client application 108 on the client device 106 may automatically and autonomously transition the display state of the client application 108 from a normal display mode where sensor glucose measurement data is invisible or hidden to a diagnostic display mode where at least some of the patient's recent sensor glucose measurement data is depicted. For example, in response to a display toggling condition, the client application 108 may automatically provide a diagnostic GUI display 2400, 2500 in lieu of a home screen GUI display 1800 that does not include any graphical representations of the patient's sensor glucose measurement data.

FIG. 24 depicts an exemplary retrospective diagnostic GUI display 2400 that includes a line chart or line graph 2404 of the patient's historical sensor glucose measurement data depicting the patient's glucose level over a preceding time period with respect to time in addition to a graphical representation 2402 of the patient's most recent sensor glucose measurement value. In this regard, the patient's most recent sensor glucose measurement value may be presented in concert with a depiction of an indication of a time associated with that measurement value or the duration that has elapsed since that most recent sensor glucose measurement value was obtained, thereby providing an indication to the patient of how recent that depicted measurement value 2402 is. In the illustrated embodiment, the line graph region of the retrospective diagnostic GUI display 2400 also includes a visually distinguishable overlay region 2406 that indicates a target range for the patient's sensor glucose measurement values. Although not illustrated in FIG. 24, in some embodiments, the line graph region of the retrospective diagnostic GUI display 2400 may also include graphical indicia of event log data in a similar manner as described above in the context of FIGS. 21-22.

In exemplary embodiments, the current sensor glucose measurement value 2402 and the historical sensor glucose graph 2404 are dynamically updated in response to receiving updated measurement data at the client device 106. For example, the monitoring device 102 may be configured to periodically sample the sensing element 104 (e.g., every minute, every five minutes, or the like), and in a real-time mode, automatically provide new measurement data samples to the client device 106 as they become available. In response to a new measurement data sample, the current sensor glucose measurement value 2402 displayed by the client application 108 is updated substantially in real-time, along with the sensor glucose graph 2404 being updated substantially concurrently to reflect the new sensor glucose measurement value corresponding to the new measurement data sample.

As described above, in one or more embodiments, the retrospective diagnostic GUI display 2400 may be presented in response to detecting a toggling condition that indicates some aspect of the patient's behavior can or should be rewarded, such as, for example, the patient obtaining new blood glucose readings with a blood glucose meter with a desired frequency, the patient engaging in exercise for a desired duration or with a desired frequency, or the like. In this regard, after transitioning to the retrospective diagnostic GUI display 2400, the client application 108 may monitor for a subsequent display toggling condition indicating the measurement data should again be hidden from the patient (e.g., task 2308). For example, the client application 108 may analyze the patient's event log data, the patient's measurement data, and/or other available information or data (e.g., the geographic location of the client device 106) to detect or otherwise identify a deviation in the patient's behavior while the retrospective diagnostic GUI display 2400 is provided that indicates depicting the measurement data could be overly influencing the patient's behavior. In response to detecting another display toggling condition, the client application 108 may automatically transition or revert to presenting the home screen GUI display 1800 on the client device 106 in lieu of the retrospective diagnostic GUI display 2400. Additionally or alternatively, the client application 108 may monitor a duration of time that has elapsed since the behavior, activity, or event being rewarded by presenting the retrospective diagnostic GUI display 2400 occurred, and automatically revert to presenting the home screen GUI display 1800 on the client device 106 once the duration of time is greater than a threshold time period. In this manner, automatically hiding the measurement data may act to stimulate or encourage desirable patient behavior with a desired frequency or duration according to the study the patient is engaged in.

FIG. 25 depicts an exemplary diagnostic GUI display 2500 that may be presented in response to detecting a toggling condition where the patient should be encouraged to engage in behavior capable of altering his or her glucose level. In this regard, the diagnostic GUI display 2500 includes a graphical representation 2502 of the patient's most recent sensor glucose measurement value along with text or other information 2504 pertaining to the patient's physiological condition. The contents of the textual information 2504 may be influenced by the patient's most recent sensor glucose measurement value or otherwise indicate the behavior being suggested or discouraged. For example, the client application 108 may automatically transition from the home screen GUI display 1800 to the diagnostic GUI display 2500 in response to detecting the most recent sensor glucose measurement value for the patient is above an upper threshold value or otherwise outside of a target range of glucose measurement values about the patient's target glucose level, with the textual information 2504 providing indication of the underlying criteria or rationale that triggered the display toggling. As another example, the client application 108 could automatically transition to the diagnostic GUI display 2500 in response to detecting the geographic location of the client device 106 is near a gym or other recreational facility or area, or in response to detecting a duration of time elapsed since a preceding exercise event exceeds a threshold duration of time. In such embodiments, the displayed textual information 2504 may be further configured to recommend or suggest exercise to the patient, a particular type of exercise, a particular duration of exercise, and/or the like to lower the patient's sensor glucose measurements to a desired range or target value. The illustrated diagnostic GUI display 2500 also includes a selectable GUI element 2506 that allows the patient to manually revert or transition back to the home screen GUI display 1800, along with a selectable GUI element 2508 that allows the patient to manually toggle or transition to a GUI display that includes a graphical representation of the patient's historical measurement data, such as retrospective GUI display 2400.

It should be noted that FIGS. 24-25 merely depict a select few exemplary diagnostic GUI displays for purposes of explanation and are not intended to be limiting. For example, in some embodiments, a prospective diagnostic GUI display including one or more predicted sensor glucose measurement values in the future presented in a line chart region concurrently to a graph of preceding historical sensor glucose measurement values could be presented to provide indication to the patient of how his or her glucose levels are expected to behave based at least in part on the patient's contemporaneous behavior and/or recent sensor glucose measurement data. In other embodiments, the diagnostic GUI display could include or otherwise be realized as a graph overlay region similar to those depicted in FIGS. 21-22 or a snapshot GUI display similar to those provided in U.S. Patent Pub. No. 2017/0106144.

By virtue of the automated display toggling described herein, patient behavior during a study may be influenced or steered in a manner that better satisfies the objectives of the study. Thus, defining the display toggling criteria or conditions to account for patient behavior in the appropriate manner may improve the usefulness or reliability of the measurement data resulting from the study.

For the sake of brevity, conventional techniques related to glucose sensing and/or monitoring, glucose regulation, communications networks, communications sessions, communications security, graphical user interfaces, menus and navigation thereof, and other functional aspects of the subject matter may not be described in detail herein. In addition, certain terminology may also be used in the herein for the purpose of reference only, and thus is not intended to be limiting. For example, terms such as “first”, “second”, and other such numerical terms referring to structures do not imply a sequence or order unless clearly indicated by the context. The foregoing description may also refer to elements or nodes or features being “connected” or “coupled” together. As used herein, unless expressly stated otherwise, “coupled” means that one element/node/feature is directly or indirectly joined to (or directly or indirectly communicates with) another element/node/feature, and not necessarily mechanically.

While at least one exemplary embodiment has been presented in the foregoing detailed description, it should be appreciated that a vast number of variations exist. It should also be appreciated that the exemplary embodiment or embodiments described herein are not intended to limit the scope, applicability, or configuration of the claimed subject matter in any way. For example, the subject matter described herein is not necessarily limited to the infusion devices and related systems described herein. Moreover, the foregoing detailed description will provide those skilled in the art with a convenient road map for implementing the described embodiment or embodiments. It should be understood that various changes can be made in the function and arrangement of elements without departing from the scope defined by the claims, which includes known equivalents and foreseeable equivalents at the time of filing this patent application. Accordingly, details of the exemplary embodiments or other limitations described above should not be read into the claims absent a clear intention to the contrary.

What is claimed is:

1. A monitoring system comprising:
a monitoring device coupled to a sensing element to obtain measurement data pertaining to a physiological condition of a patient;
a remote device coupled to a communications network; and
a monitoring application at a client device coupled to the communications network to monitor a first network for a data ready indication from the monitoring device, establish a communications session with the monitoring device on the first network in response to the data ready indication, receive the measurement data from the monitoring device over the first network, upload the measurement data to the remote device over the communications network, and automatically toggle display of at least a portion of the measurement data at the monitoring device in response to detecting a toggling condition.

2. The monitoring system of claim 1, the monitoring application providing one or more graphical user interface displays on the client device including graphical user interface elements for defining an event related to the physiological condition, wherein:
the client device includes a user input device for receiving indicia of an event type and descriptive information associated with the event via the graphical user interface elements; and
the monitoring application automatically toggles display of at least the portion of the measurement data at the monitoring device in response to the event.

3. The monitoring system of claim 1, wherein the monitoring application automatically toggles display of at least the portion of the measurement data at the monitoring device by selectively displaying the portion of the measurement data in a manner that is influenced by a behavior of the patient.

4. The monitoring system of claim 1, wherein:
the sensing element comprises an interstitial glucose sensing element providing the measurement data comprising glucose levels sensed by the interstitial glucose sensing element; and
the monitoring application detects the toggling condition based at least in part on the glucose levels sensed by the interstitial glucose sensing element.

5. A method of monitoring a physiological condition of a patient, the method comprising:
obtaining, at a computing device, measurement data for the physiological condition of the patient;
providing, at the computing device, a graphical user interface display pertaining to monitoring the physiological condition of the patient, wherein the measurement data is hidden from the graphical user interface display;
identifying, at the computing device, a toggling condition for the graphical user interface display while the graphical user interface display is presented at the computing device;
in response to the toggling condition, automatically providing, at the computing device, a graphical representation of at least some of the measurement data; and
after providing the graphical representation of at least some of the measurement data:
identifying, at the computing device, a second toggling condition while the graphical representation of at least some of the measurement data is presented at the computing device; and
automatically removing the graphical representation of at least some of the measurement data from presentation at the computing device in response to the second toggling condition.

6. The method of claim 5, further comprising:
automatically configuring a monitoring device coupled to a sensing element to automatically provide updated measurement data for the physiological condition of the patient from the sensing element to the computing device in response to the toggling condition; and
dynamically updating, at the computing device, the graphical representation of at least some of the measurement data to reflect the updated measurement data prior to identifying the second toggling condition.

7. The method of claim 6, wherein:
automatically configuring the monitoring device comprises the computing device instructing the monitoring device to enter a real-time mode; and
the monitoring device automatically sets a data ready flag in an advertisement packet of a message communicated on a wireless network to true when a new measurement data sample is available in the real-time mode.

8. The method of claim 7, the monitoring device setting the data ready flag to true when an amount of the measurement data is greater than a threshold prior to entering the real-time mode, wherein obtaining the measurement data comprises:
establishing, by the computing device, a communications session with the monitoring device on the wireless network in response to detecting the data ready flag is true;
receiving, by the computing device, the measurement data from the monitoring device over the wireless network; and
uploading, by the computing device, the measurement data to a remote device on a second network.

9. The method of claim 5, further comprising:
obtaining, at the computing device, event log data via a user input device associated with the computing device; and
identifying the toggling condition based at least in part on the event log data.

10. The method of claim 9, wherein identifying the toggling condition comprises identifying a meal event based at least in part on the event log data.

11. The method of claim 5, wherein identifying the toggling condition comprises identifying an exercise event.

12. The method of claim 5, wherein identifying the second toggling condition comprises identifying a deviation in behavior by the patient.

13. The method of claim 5, wherein identifying the second toggling condition comprises identifying an absence of a blood glucose measurement within a threshold period of time.

14. The method of claim 5, wherein identifying the toggling condition comprises determining a geographic location of the computing device is within a threshold distance of a location of interest.

15. The method of claim 5, wherein identifying the toggling condition comprises detecting the toggling condition when one or more measurement data samples of the measurement data violates a threshold value.

16. The method of claim 5, wherein identifying the second toggling condition comprises detecting the second toggling condition when one or more measurement data samples is within a desired measurement range.

17. The method of claim 5, wherein:
obtaining the measurement data comprises:
establishing, by the computing device, a communications session with a monitoring device over a wireless network; and
receiving, by the computing device, the measurement data from the monitoring device over the wireless network via the communications session; and
the monitoring device comprises a continuous glucose monitor coupled to an interstitial glucose sensing element inserted into the patient; and
the measurement data comprises glucose levels sensed by the interstitial glucose sensing element.

18. The method of claim 5, wherein:
identifying the toggling condition comprises identifying a patient behavior to be rewarded; and
automatically providing the graphical representation of at least some of the measurement data further comprises displaying content rewarding the patient behavior at the computing device.

19. A method of monitoring a physiological condition of a patient, the method comprising:
pairing a client computing device and a monitoring device over a personal area network, the monitoring device being coupled to a sensing element obtaining measurement data for the physiological condition of the patient;
providing, at the client computing device, a home screen graphical user interface display pertaining to monitoring the physiological condition of the patient, wherein the measurement data is hidden from the home screen graphical user interface display;
identifying, at the client computing device, a first display toggling condition while the home screen graphical user interface display is presented at the client computing device;
in response to the first display toggling condition:
automatically establishing, by the client computing device, a communications session with the monitoring device on the personal area network;
receiving, by the client computing device, the measurement data from the monitoring device via the communications session; and
automatically providing, at the client computing device, a diagnostic display including a graphical representation of the measurement data in lieu of the home screen graphical user interface display; and
identifying, at the client computing device, a second display toggling condition while the diagnostic display is presented at the client computing device; and
automatically presenting the home screen graphical user interface display in lieu of the diagnostic display at the client computing device in response to the second display toggling condition.

20. The method of claim 19, the home screen graphical user interface display including one or more graphical user interface elements for receiving event log data from the patient, wherein identifying the first display toggling condition comprises identifying the first display toggling condition based at least in part on the event log data.
